(19) United States
(12) Patent Application Publication
(10) Pub. No.: US 2025/0057819 A1
(43) Pub. Date: Feb. 20, 2025

(54) DRUG COMBINATIONS FOR INHIBITING CORONAVIRUS REPLICATION

(71) Applicant: INSTITUT NATIONAL DE LA RECHERCHE SCIENTIFIQUE, Quebec (CA)

(72) Inventors: Steven LAPLANTE, Laval (CA); Richard Boulon, Montreal (CA); Clément Mazeaud, Montreal (CA); Alain Lamarre, Laval (CA); Laurent Chatel-Chaix, Montreal (CA)

(21) Appl. No.: 18/719,122

(22) PCT Filed: Dec. 22, 2022
(86) PCT No.: PCT/CA2022/051888

§ 371 Date: Jun. 12, 2024

Related U.S. Application Data
(60) Provisional application No. 63/293,433, filed on Dec. 23, 2021.

(51) Int. Cl. A61K 31/435; A61K 31/055; A61K 31/135; A61K 31/138; A61K 31/426; A61K 31/496; A61K 31/498; A61K 31/5377; A61K 31/5415; A61K 31/58; A61K 31/593; A61K 31/675; A61K 31/7068; A61P 31/14

(52) U.S. Cl. CPC A61K 31/435; A61K 31/055; A61K 31/135; A61K 31/138; A61K 31/426; A61K 31/496; A61K 31/498; A61K 31/5377; A61K 31/5415; A61K 31/58; A61K 31/593; A61K 31/675; A61K 31/7068; A61P 31/14

(57) ABSTRACT

A combination comprising tilorone/nelfinavir; tilorone/molnupinavir; tilorone/remdesivir; tilorone/alfacalcidol: tilorone/clofazimine; tilorone/danazol; tilorone/manidipine; tilorone/sertraline; tilorone/trifluoperazine; remdesivir/hexachlorophene; remdesivir/mycophenolate; mycophenolate/hexachlorophene; mycophenolate/nelfinavir; or mycophenolate/nitazoxanide. This combination can be used for blocking the replication of a coronavirus in a coronavirus-infected cell, for treating an infection by a coronavirus in a subject, for treating a coronavirus-related disease in a subject, and/or for reducing the risk of developing a coronavirus-related disease or the severity of a coronavirus-related disease in a subject.

### CROSS REFERENCE TO RELATED APPLICATIONS

This application claims benefit, under 35 U.S.C. § 119(e), of U.S. provisional application Ser. No. 63/293,433, filed on Dec. 23, 2022. All documents above are incorporated herein in their entirety by reference.

### FIELD OF THE INVENTION

The present disclosure generally relates to viral infection, and more particularly to the prevention and/or treatment of coronavirus infection such as severe acute respiratory syndrome coronavirus 2 (SARS-CoV-2) infection.

### BACKGROUND OF THE INVENTION

Coronaviruses are responsible for a wide spectrum of respiratory and gastrointestinal diseases in wild and domestic animals along with humans (1). In general, six members of the coronavirus family are capable of infecting humans. Four of these circulate worldwide, causing the common cold, while the remaining two have been implicated in severe acute respiratory syndrome (SARS) and the Middle East respiratory syndrome (MERS-CoV) (2-5). Recently, a new type of coronavirus designated SARS-CoV-2 was found to be the cause of SARS (including fever, dyspnea, asthenia and pneumonia) and triggered the pandemic known as COVID-19 (6). Given this, it is becoming more apparent that we must augment our arsenal of small-molecule drugs to combat coronaviruses.

However, it is well known that the process of finding new drugs (if successful) is expected to take between 10-15 years, which is simply too long when humankind is impacted by a rapid-onset pandemic. Although securing vaccines is expected to take 8-12 years, significant breakthroughs in technologies contributed to the surprisingly fast discovery of multiple COVID-19 vaccine options (7-8). Despite this breakthrough, the emergence of viral variants has resulted in wave after wave of infections, and subsets of populations remain unvaccinated for multiple reasons. Thus, new anti-viral options are urgently needed to continue building our arsenal.

One alternative approach for fast-tracking small-molecule drug discovery is “drug repurposing”. This strategy involves the screening of marketed drugs that have already been approved for another indication, hoping to discover new applications to treat additional diseases. A major advantage of repurposing is that an approved drug that has already cleared various hurdles can therefore advance much more rapidly to later phases in clinical trials for the new indication. This significantly reduces time, costs and risks. To date, cell-culture screens have identified numerous drugs that are active against SARS-CoV-2, and have resulted in limited successes in clinical trials (9-12). In general, the mechanism of action of many of the other hits are complex and poorly understood (13-15). Furthermore, the cell-culture screening methods are lengthy and difficult.

The more promising approach involved mechanism-based drugs (e.g. remdesivir)⁷ that are likely active against protein classes such as polymerases and proteases. Some examples include Lopinavir (+/−ritonavir) (3CLpro/Mpro). Reports from in vitro studies suggest an EC₅₀ of 26 μM for Lopinavir (16). However, no benefit was observed with the treatment in adults hospitalized with Covid-19 (17). Fortunately, two promising drugs have recently emerged. Nirmatrelvir is an antiviral drug developed by Pfizer which acts as an orally active 3CL protease inhibitor. The combination of Nirmatrelvir with ritonavir is in phase III trials for the treatment of COVID-19 (18-21). The combination is expected to be marketed under the brand name Paxlovid. Molnupiravir, sold under the brand name Lagevrio among others, is an antiviral medication that inhibits the replication of certain RNA viruses, and is used to treat COVID-19 in those infected by SARS-CoV-2. Molnupiravir is a prodrug of the synthetic nucleoside derivative N4-hydroxycytidine and exerts its antiviral action through the introduction of copying errors during viral RNA replication (22-23).

### SUMMARY OF THE INVENTION

In accordance with the present invention, there is provided:
1. A combination of drugs comprising:
   A) tilorone or a pharmaceutically acceptable salt thereof and B) nelfinavir or a pharmaceutically acceptable salt thereof,
   A) tilorone or a pharmaceutically acceptable salt thereof and B) molnupinavir or a pharmaceutically acceptable salt thereof,
   A) tilorone or a pharmaceutically acceptable salt thereof and B) remdesivir or a pharmaceutically acceptable salt thereof,
   A) tilorone or a pharmaceutically acceptable salt thereof and B) alfacalcidol or a pharmaceutically acceptable salt thereof,
   A) tilorone or a pharmaceutically acceptable salt thereof and B) clofazimine or a pharmaceutically acceptable salt thereof,
   A) tilorone or a pharmaceutically acceptable salt thereof and B) danazol or a pharmaceutically acceptable salt thereof,
   A) tilorone or a pharmaceutically acceptable salt thereof and B) manidipine or a pharmaceutically acceptable salt thereof,
   A) tilorone or a pharmaceutically acceptable salt thereof and B) sertraline or a pharmaceutically acceptable salt thereof,
   A) tilorone or a pharmaceutically acceptable salt thereof and B) trifluoperazine or a pharmaceutically acceptable salt thereof,
   A) remdesivir or a pharmaceutically acceptable salt thereof and B) hexachlorophene or a pharmaceutically acceptable salt thereof,
   A) remdesivir or a pharmaceutically acceptable salt thereof and B) mycophenolate or a pharmaceutically acceptable salt thereof,
   A) mycophenolate or a pharmaceutically acceptable salt thereof and B) hexachlorophene or a pharmaceutically acceptable salt thereof,
   A) mycophenolate or a pharmaceutically acceptable salt thereof and B) nelfinavir or a pharmaceutically acceptable salt thereof, or
   A) mycophenolate or a pharmaceutically acceptable salt thereof and B) nitazoxanide or a pharmaceutically acceptable salt thereof.
2. The combination of embodiment 1, comprising:
   A) tilorone or a pharmaceutically acceptable salt thereof and B) nelfinavir or a pharmaceutically acceptable salt thereof,
   A) tilorone or a pharmaceutically acceptable salt thereof and B) molnupinavir or a pharmaceutically acceptable salt thereof, or
   A) tilorone or a pharmaceutically acceptable salt thereof and B) remdesivir or a pharmaceutically acceptable salt thereof.
3. The combination of embodiment 2, comprising:
   A) tilorone or a pharmaceutically acceptable salt thereof and B) nelfinavir or a pharmaceutically acceptable salt thereof, or
   A) tilorone or a pharmaceutically acceptable salt thereof and B) molnupinavir or a pharmaceutically acceptable salt thereof.
4. The combination of embodiment 1, comprising (A) tilorone or a pharmaceutically acceptable thereof and (B) nelfinavir or a pharmaceutically acceptable thereof.
5. The combination of embodiment 1, comprising (A) tilorone or a pharmaceutically acceptable thereof and (B) remdesivir or a pharmaceutically acceptable thereof.
6. The combination of embodiment 1, comprising (A) tilorone or a pharmaceutically acceptable thereof and (B) molnupinavir or a pharmaceutically acceptable thereof.
7. The combination of embodiment 1, comprising A) tilorone or a pharmaceutically acceptable salt thereof and B) alfacalcidol or a pharmaceutically acceptable salt thereof,
8. The combination of embodiment 1, comprising A) tilorone or a pharmaceutically acceptable salt thereof and B) clofazimine or a pharmaceutically acceptable salt thereof,
9. The combination of embodiment 1, comprising A) tilorone or a pharmaceutically acceptable salt thereof and B) danazol or a pharmaceutically acceptable salt thereof,
10. The combination of embodiment 1, comprising A) tilorone or a pharmaceutically acceptable salt thereof and B) manidipine or a pharmaceutically acceptable salt thereof,
11. The combination of embodiment 1, comprising A) tilorone or a pharmaceutically acceptable salt thereof and B) sertraline or a pharmaceutically acceptable salt thereof,
12. The combination of embodiment 1, comprising A) tilorone or a pharmaceutically acceptable salt thereof and B) trifluoperazine or a pharmaceutically acceptable salt thereof,
13. The combination of embodiment 1, comprising A) remdesivir or a pharmaceutically acceptable salt thereof and B) hexachlorophene or a pharmaceutically acceptable salt thereof,
14. The combination of embodiment 1, comprising A) remdesivir or a pharmaceutically acceptable salt thereof and B) mycophenolate or a pharmaceutically acceptable salt thereof,
15. The combination of embodiment 1, comprising A) mycophenolate or a pharmaceutically acceptable salt thereof and B) hexachlorophene or a pharmaceutically acceptable salt thereof,
16. The combination of embodiment 1, comprising A) mycophenolate or a pharmaceutically acceptable salt thereof and B) nelfinavir or a pharmaceutically acceptable salt thereof, or
17. The combination of embodiment 1, comprising A) mycophenolate or a pharmaceutically acceptable salt thereof and B) nitazoxanide or a pharmaceutically acceptable salt thereof.
18. The combination of any one or embodiments 1 to 17, wherein the pharmaceutically acceptable salt of nelfinavir is nelfinavir methanesulfonate.
19. The combination of any one of embodiments 1 to 17, wherein the pharmaceutically acceptable salt of tilorone is tilorone dihydrochloride.
20. The combination of any one of embodiments 1 to 17, wherein the pharmaceutically acceptable salt of manidipine is manidipine dihydrochloride.
21. The combination of any one of embodiments 1 to 17, wherein the pharmaceutically acceptable salt of sertraline is sertraline hydrochloride.
22. The combination of any one of embodiments 1 to 17, wherein the pharmaceutically acceptable salt of the trifluoperazine is trifluoperazine dihydrochloride.
23. The combination of any one of embodiments 1 to 17, wherein the pharmaceutically acceptable salt of mycophenolate is mycophenolate mofetil.
24. The combination of any one of embodiments 1 to 23, wherein components (a) and (b) are formulated in a same pharmaceutical composition.
25. The combination of any one of embodiments 1 to 23, wherein components (a) and (b) are formulated in different pharmaceutical compositions.
26. A pharmaceutical composition comprising the combination of any one of embodiments 1 to 20 and one or more pharmaceutically acceptable excipient.
27. A method for blocking the replication of a coronavirus in a coronavirus-infected cell, comprising contacting the cell with an effective amount of the combination of any one of embodiments 1 to 26.
28. A method for treating an infection by a coronavirus in a subject comprising administering to said subject an effective amount of the combination of any one of embodiments 1 to 26.
29. A method for treating a coronavirus-related disease in a subject comprising administering to said subject an effective amount of the combination of any one of embodiments 1 to 26.
30. A method for reducing the risk of developing a coronavirus-related disease or the severity of a coronavirus-related disease in a subject comprising administering to said subject an effective amount of the combination of any one of embodiments 1 to 26.
31. The method of any one of embodiments 27 to 30, wherein the coronavirus is a beta-coronavirus.
32. The method of embodiment 31, wherein the beta-coronavirus is SARS-CoV-2.
33. The method of embodiment 29, wherein the coronavirus-related disease is COVID-19.
34. The method of any one of embodiments 28 to 30, wherein the subject is a human.
35. Use of the combination of any one of embodiments 1 to 26, for the manufacture of a medicament for blocking the replication of a coronavirus in a coronavirus-infected cell.
36. Use of the combination of any one of embodiments 1 to 26, for the manufacture of a medicament for treating an infection by a coronavirus in a subject.
37. Use of the combination of any one of embodiments 1 to 26, for the manufacture of a medicament for treating a coronavirus-related disease in a subject.
38. Use of the combination of any one of embodiments 1 to 26, for the manufacture of a medicament for reducing the risk of developing a coronavirus-related disease or the severity of a coronavirus-related disease in a subject.
39. The use of any one of embodiments 35 to 38, wherein the coronavirus is a beta-coronavirus.
40. The use of embodiment 39, wherein the beta-coronavirus is SARS-CoV-2.
41. The use of embodiment 38, wherein the coronavirus-related disease is COVID-19.
42. The use of any one of embodiments 36 to 38, wherein the subject is a human.
43. The combination of any one of embodiments 1 to 26 for use in blocking the replication of a coronavirus in a coronavirus-infected cell.
44. The combination of any one of embodiments 1 to 26 for use in treating an infection by a coronavirus in a subject.
45. The combination of any one of embodiments 1 to 26 for use in treating a coronavirus-related disease in a subject.
46. The combination of any one of embodiments 1 to 26 for use in reducing the risk of developing a coronavirus- related disease or the severity of a coronavirus-related disease in a subject.
47 The combination for use according to any one of embodiments 43 to 46, wherein the coronavirus is a beta- coronavirus.
48. The combination for use according to embodiment 47, wherein the beta-coronavirus is SARS-CoV-2.
49. The combination for use according to embodiment 45, wherein the coronavirus-related disease is COVID-19.
50. The combination for use according to any one of embodiments 44 to 46, wherein the subject is a human.

### BRIEF DESCRIPTION OF THE DRAWINGS

In the appended drawings:

FIG. 1A. Screening and synergy study methods used in Example 1.

FIGS. 1B and C. Screening representation of the results of the 676 drugs tested against rHCoVs-OC43 (B and C, full and empty dots), 65 drugs identified with promising activity (B, filled circles) and 50 drugs were characterized (C, filled circles).

FIG. 2. Identification of several compounds with antiviral activity against rHCoVs-OC43. Dose-response curves of selected compounds from the 10 best drugs against rHCoVs-OC43. HRT-18 cells were infected with rHCoVs-OC43 virus at MOI of 0.025, during 4 hours, and treated for 48 hours with a complete range of drugs (0.5, 1, 5, 10, 50, 100, 500, 1 000, 5 000 or 10 000 nM). The antiviral activity was measured by the decrease of R-Luc assay (squares) and the cellular viability by MTT test (circles). Data represents the mean+/−standard deviation (error bars) of the results of minimum three independent experiment.

FIG. 3 shows the developed custom version of a checkerboard synergy assay used in Example 1.

FIG. 4. Method to identify an antagonism combination of drugs (chloroquine and remdesivir). HRT-18 cells were infected with rHCoVs-OC43 virus at MOI of 0.025, during 4 hours, and treated for 48 hours with a complete range of chloroquine with or without remdesivir. Chloroquine and remdesivir combinations in fast-screening study, representations as a curve (A). The antiviral activity was measured by the decrease of R-Luc assay (square, diamond) and the cellular viability by MTT test (filled circles, diamonds) with (empty circles, diamonds) or without (filled circles, squares) 20 nM of remdesivir. Chloroquine and remdesivir combinations in fast-screening study, representations as a HSA interactive 3D surface representation (B). Chloroquine and remdesivir combinations in matrix study, representation of antiviral activity as a curve (C) or HSA interactive 3D surface representation (D) and viability as a curve (E). HRT-18 cells were infected with rHCoVs-OC43 virus at MOI of 0.025, during 4 hours, and treated for 48 hours with two complete range of chloroquine or remdesivir (drug concentrations: 0; 0.001; 0.01; 0.1; 1 μM). Data represent the mean+/−standard deviation (error bars) of the results of minimum three independent experiment. FIG. 5. Method to identify a synergistic combination of drugs (nitazoxanide and remdesivir). HRT-18 cells were infected with rHCoVs-OC43 virus at MOI of 0.025, during 4 hours, and treated for 48 hours with a complete range of nitazoxanide with 20 nM of remdesivir. Nitazoxanide and remdesivir combinations in fast-screening study, representations as a curve (A). The antiviral activity was measured by the decrease of R-Luc assay (black square, red diamond) and the cellular viability by MTT test (blue circle, black diamond) with (empty circle, diamond) or without (filled circle, black square) 20 nM of remdesivir. Nitazoxanide and remdesivir combinations in fast-screening study, representations as a HSA interactive 3D surface representation (B). Nitazoxanide and remdesivir combinations in matrix study, representation of antiviral activity as a curve (C) or HSA interactive 3D surface representation (D) and viability as a curve (E). HRT-18 cells were infected with rHCoVs-OC43 virus at MOI of 0.025, during 4 hours, and treated for 48 hours with two complete range of nitazoxanide or remdesivir (drug concentrations: 0; 0.001; 0.01; 0.1; 1 μM). Data represent the mean+/−standard deviation (error bars) of the results of minimum three independent experiment.

FIG. 6. Identification of several synergistic drug combinations in rHCoVs-OC43. HSA interactive 3D surface representation of tilorone and remdesivir (A), nitazoxanide and remdesivir (B), trifluoperazine and tilorone (C), nelfinavir and tilorone (D). Data represent the mean of the results of minimum three independent experiments.

FIG. 7. Efficacy and toxicity of best SARS-CoV-2 drugs in Huh7.5 (black) and Calu3 (red) cells in monotherapy. Huh7.5 (squares) or Calu3 (diamonds) cells were infected with SARS-CoV-2 virus at MOI of 0.05, during 2 hours, and treated for 48 hours with a complete range of drugs (0.0001, 0.001, 0.01, 0.1, 1, or 10 μM). The SARS-CoV-2 viral concentration was measured by plate assay method in Vero E6 cells and the cellular viability by MTT test. Data represent the mean+/−standard deviation (error bars) of the results of minimum three independent experiments.

FIG. 8. SARS-CoV-2 drugs combination studies in Huh7.5 cells. HSA interactive 3D surface representation of drug combination in Huh7.5 cells; (A) nelfinavir and tilorone, (B) molnupiravir and tilorone, (C) Paxlovid and tilorone, (D) trifluoperazine and tilorone. Data represent the mean of the results of minimum three independent experiments.

FIG. 9: SARS-CoV-2 drugs combination studies Calu3 cells. HSA interactive 3D surface representation of drug combination in Calu3 cells; (A) nelfinavir and tilorone, (B) molnupiravir and tilorone, and (C) Paxlovid and tilorone. Data represent the mean of the results of minimum three independent experiments.

FIG. 10. Efficacy of best SARS-CoV-2 drugs in monotherapy against SARS-CoV-2 vido (black square), SARS-CoV-2 Alpha/UK (diamond) and SARS-CoV-2 Beta/SA (circle) strains. Huh7.5 cells were infected with SARS-CoV-2 variants at MOI of 0.05, during 2 hours, and treated for 48 hours with a complete range of drugs (0.0001, 0.001, 0.01, 0.1, 1, or 10 μM) with (A) tilorone, (B) nelfinavir, (C) Paxlovid, and (D) molnupinavir. The SARS-CoV-2 viral concentration was measured by plate assay method in Vero E6. Data represent the mean+/−standard deviation (error bars) of the results of minimum three independent experiments.

FIG. 11. Method to identify an antagonism (chloroquine and remdesivir, A-C) or synergistic (nitazoxanide and remdesivir, D-F) combination of drugs. Representations as a curve of chloroquine and remdesivir (C) or nitazoxanide-remdesivir (F) combinations in fast-screening study. HRT-18 cells were infected with rHCoVs-OC43 virus at MOI of 0.025, during 4 hours, and treated for 48 hours with a complete range of drugs (chloroquine or nitazoxanide) with or without remdesivir. The antiviral activity was measured by the decrease of R-Luc assay with (empty circles) or without (squares) 20 nM of remdesivir. Chloroquine-remdesivir or nitazoxanide-remdesivir combinations in matrix study, representation of antiviral activity as a curve (B/E). HRT-18 cells were infected with rHCoVs-OC43 virus at

MOI of 0.025, during 4 hours, and treated for 48 hours with two complete range of drugs (chloroquine or nitazoxanide) and remdesivir (drug concentrations: 0; 0.001; 0.01; 0.1; 1 μM). The antiviral activity was measured by the decrease of R-Luc assay. Data represent the mean+/−standard deviation (error bars) of the results of minimum three independent experiment.

FIG. 12 shows in-house first results in rHCoVs-OC43assay: method validation, increase number of concentration points dose-response data and analyses for the nitazoxanide-remdesivir synergistic combination shown as (A) HAS interactive table, (B) 3D surface representation. HRT-18 cells were infected with rHCoVs-OC43 virus at MOI of 0.025, during 4 hours, and treated for 48 hours with complete range of drugs (0.5, 1, 5, 10, 50, 100, 500, 1 000, 5 000 or 10 000 nM) and remdesivir (drug concentrations: 0; 0.001; 0.01; 0.1; 1 μM). The antiviral activity was measured by the decrease of R-Luc assay. Data represent the mean+/−standard deviation (error bars) of the results of minimum three independent experiment.

FIG. 13 shows synergy matrix results of remdesivir with chloroquine in rHCoVs-OC43assay (first compound tested). HRT-18 cells were infected with rHCoVs-OC43 virus at MOI of 0.025, during 4 hours, and treated for 48 hours with complete range of chloroquine (0.5, 1, 5, 10, 50, 100, 500, 1 000, 5 000 or 10 000 nM) and remdesivir (drug concentrations: 0; 0.001; 0.01; 0.1; 1 μM). The antiviral activity was measured by the decrease of R-Luc assay. Data represent the mean+/−standard deviation (error bars) of the results of minimum three independent experiment.

FIG. 14 shows synergy matrix results of remdesivir with clofazimine in rHCoVs-OC43assay (first compound tested). HRT-18 cells were infected with rHCoVs-OC43 virus at MOI of 0.025, during 4 hours, and treated for 48 hours with complete range of clofazimine (0.5, 1, 5, 10, 50, 100, 500, 1 000, 5 000 or 10 000 nM) and remdesivir (drug concentrations: 0; 0.001; 0.01; 0.1; 1 μM). The antiviral activity was measured by the decrease of R-Luc assay. Data represent the mean+/−standard deviation (error bars) of the results of minimum three independent experiment.

FIG. 15 shows synergy matrix results of remdesivir with tilorone in rHCoVs-OC43assay. HRT-18 cells were infected with rHCoVs-OC43 virus at MOI of 0.025, during 4 hours, and treated for 48 hours with complete range of tilorone (0.5, 1, 5, 10, 50, 100, 500, 1 000, 5 000 or 10 000 nM) and remdesivir (drug concentrations: 0; 0.001; 0.01; 0.1; 1 μM). The antiviral activity was measured by the decrease of R-Luc assay. Data represent the mean+/−standard deviation (error bars) of the results of minimum three independent experiment.

FIG. 16 shows synergy matrix results of remdesivir with nitazoxanide in rHCoVs-OC43 assay. HRT-18 cells were infected with rHCoVs-OC43 virus at MOI of 0.025, during 4 hours, and treated for 48 hours with complete range of nitazoxanide (0.5, 1, 5, 10, 50, 100, 500, 1 000, 5 000 or 10 000 nM) and remdesivir (drug concentrations: 0; 0.001; 0.01; 0.1; 1 μM). The antiviral activity was measured by the decrease of R-Luc assay. Data represent the mean+/−standard deviation (error bars) of the results of minimum three independent experiment.

FIG. 17 shows the synergy matrix results of tilorone with trifluoperazine in rHCoVs-OC43 assay. HRT-18 cells were infected with rHCoVs-OC43 virus at MOI of 0.025, during 4 hours, and treated for 48 hours with complete range of trifluoperazine (0.5, 1, 5, 10, 50, 100, 500, 1 000, 5 000 or 10 000 nM) and tilorone (drug concentrations: 0; 0.001; 0.01; 0.1; 1 μM). The antiviral activity was measured by the decrease of R-Luc assay. Data represent the mean+/−standard deviation (error bars) of the results of minimum three independent experiment.

FIG. 18 shows the synergy matrix results of tilorone with nelfinavir in rHCoVs-OC43 assay. HRT-18 cells were infected with rHCoVs-OC43 virus at MOI of 0.025, during 4 hours, and treated for 48 hours with complete range of nelfinavir (0.5, 1, 5, 10, 50, 100, 500, 1 000, 5 000 or 10 000 nM) and tilorone (drug concentrations: 0; 0.001; 0.01; 0.1; 1 μM). The antiviral activity was measured by the decrease of R-Luc assay. Data represent the mean+/−standard deviation (error bars) of the results of minimum three independent experiment.

FIG. 19 shows the synergy matrix results of tilorone with nelfinavir in SARS-CoV-2 assay as HSA 3D surface representation (A), interactive table (B) and as a curve (D). Efficacy of nelfinavir against SARS-CoV-2 replication representation as a curve (C). C. Huh7.5 cells were infected with SARS-CoV-2 virus at MOI of 0.05, during 2 hours, and treated for 48 hours with a complete range of nelfinavir (0.0001, 0.001, 0.01, 0.1, 1, or 10 μM). ABD. Huh7.5 cells were infected with SARS-CoV-2 virus at MOI of 0.05, during 2 hours, and treated for 48 hours with a complete range of nelfinavir (0.0001, 0.001, 0.01, 0.1, 1, or 10 μM) and 0 μM (square), 0.01 μM (filled circle), 0.1 μM (diamond) or 1 μM (empty circle) of tilorone. The SARS-CoV-2 viral concentration was measured by plate assay method in Vero E6. Data represent the mean+/−standard deviation (error bars) of the results of minimum three independent experiments.

### DETAILED DESCRIPTION OF THE INVENTION

Turning now to the invention in more details, there is provided a combination of drugs that comprises:
A) tilorone or a pharmaceutically acceptable salt thereof and B) nelfinavir or a pharmaceutically acceptable salt thereof,
A) tilorone or a pharmaceutically acceptable salt thereof and B) molnupinavir or a pharmaceutically acceptable salt thereof,
A) tilorone or a pharmaceutically acceptable salt thereof and B) remdesivir or a pharmaceutically acceptable salt thereof,
A) tilorone or a pharmaceutically acceptable salt thereof and B) alfacalcidol or a pharmaceutically acceptable salt thereof,
A) tilorone or a pharmaceutically acceptable salt thereof and B) clofazimine or a pharmaceutically acceptable salt thereof,
A) tilorone or a pharmaceutically acceptable salt thereof and B) danazol or a pharmaceutically acceptable salt thereof,
A) tilorone or a pharmaceutically acceptable salt thereof and B) manidipine or a pharmaceutically acceptable salt thereof,
A) tilorone or a pharmaceutically acceptable salt thereof and B) sertraline or a pharmaceutically acceptable salt thereof,
A) tilorone or a pharmaceutically acceptable salt thereof and B) trifluoperazine or a pharmaceutically acceptable salt thereof,
A) remdesivir or a pharmaceutically acceptable salt thereof and B) hexachlorophene or a pharmaceutically acceptable salt thereof,
A) remdesivir or a pharmaceutically acceptable salt thereof and B) mycophenolate or a pharmaceutically acceptable salt thereof,
A) mycophenolate or a pharmaceutically acceptable salt thereof and B) hexachlorophene or a pharmaceutically acceptable salt thereof,
A) mycophenolate or a pharmaceutically acceptable salt thereof and B) nelfinavir or a pharmaceutically acceptable salt thereof, or
A) mycophenolate or a pharmaceutically acceptable salt thereof and B) nitazoxanide or a pharmaceutically acceptable salt thereof.

In preferred embodiments, the combination of drugs comprises A) tilorone or a pharmaceutically acceptable salt thereof and:
B) nelfinavir or a pharmaceutically acceptable salt thereof,
B) molnupinavir or a pharmaceutically acceptable salt thereof, or
B) remdesivir or a pharmaceutically acceptable salt thereof.

Indeed, based on the data provided in the Examples below, the above drug combinations are unexpectedly synergistic, i.e., have an effect greater than the sum of their individual effects, when used against coronaviruses. As reported below, the present inventors have considered combinations of the 676 different drugs (i.e., more than 450 000 potential combinations) to arrive at the above drug combinations.

In preferred embodiments, the combination comprises A) tilorone or a pharmaceutically acceptable salt thereof and:
B) nelfinavir or a pharmaceutically acceptable salt thereof, or
B) molnupinavir or a pharmaceutically acceptable salt thereof.

In preferred embodiments, the combination comprises A) tilorone or a pharmaceutically acceptable salt thereof and B) nelfinavir or a pharmaceutically acceptable salt thereof.

In alternative embodiments, the combination comprises A) tilorone or a pharmaceutically acceptable salt thereof and B) molnupinavir or a pharmaceutically acceptable salt thereof.

In alternative embodiments, the combination comprises A) tilorone or a pharmaceutically acceptable salt thereof and B) remdesivir or a pharmaceutically acceptable salt thereof.

In alternative embodiments, the combination comprises A) tilorone or a pharmaceutically acceptable salt thereof and B) alfacalcidol or a pharmaceutically acceptable salt thereof.

In alternative embodiments, the combination comprises A) tilorone or a pharmaceutically acceptable salt thereof and B) clofazimine or a pharmaceutically acceptable salt thereof.

In alternative embodiments, the combination comprises A) tilorone or a pharmaceutically acceptable salt thereof and B) danazol or a pharmaceutically acceptable salt thereof.

In alternative embodiments, the combination comprises A) tilorone or a pharmaceutically acceptable salt thereof and B) manidipine or a pharmaceutically acceptable salt thereof,

In alternative embodiments, the combination comprises A) tilorone or a pharmaceutically acceptable salt thereof and B) sertraline or a pharmaceutically acceptable salt thereof.

In alternative embodiments, the combination comprises A) tilorone or a pharmaceutically acceptable salt thereof and B) trifluoperazine or a pharmaceutically acceptable salt thereof.

In alternative embodiments, the combination comprises A) remdesivir or a pharmaceutically acceptable salt thereof and B) hexachlorophene or a pharmaceutically acceptable salt thereof.

In alternative embodiments, the combination comprises A) remdesivir or a pharmaceutically acceptable salt thereof and B) mycophenolate or a pharmaceutically acceptable salt thereof.

In alternative embodiments, the combination comprises A) mycophenolate or a pharmaceutically acceptable salt thereof and B) hexachlorophene or a pharmaceutically acceptable salt thereof.

In alternative embodiments, the combination comprises A) mycophenolate or a pharmaceutically acceptable salt thereof and B) nelfinavir or a pharmaceutically acceptable salt thereof.

In alternative embodiments, the combination comprises A) mycophenolate or a pharmaceutically acceptable salt thereof and B) nitazoxanide or a pharmaceutically acceptable salt thereof.

The term “pharmaceutically acceptable salts” means salts of the above-noted compounds which retain pharmacological activities of interest, i.e., activities to inhibit coronavirus replication, and which are not toxic. These salts may be formed using inorganic acids such as hydrochloride, hydrobromide and hydroiodide, or organic acids such as acetate, adipate, alginate, aspartate, benzoate, benzenesulfonate, p-toluenesulfonate, bisulfate, sulfamate, sulfate, naphthylate, butyrate, citrate, camphorate, camphosulfate, cyclopentanepropionate, digluconate, dodecylsulfate, ethanesulfate, fumarate, glucoheptanoate, glycerophosphate, hemisulfate, heptanoate, hexanoate, 2- hydroxyethanesulfate, lactate, maleate, methanesulfonate, 2-naphthalenesulfonate, nicotinate, oxalate, tosylate and undecanoate.

In an embodiment, the nelfinavir is in the form of a pharmaceutically acceptable salt, preferably nelfinavir methane sulfonate.

In an embodiment, the tilorone is in the form of a pharmaceutically acceptable salt, preferably tilorone dihydrochloride.

In an embodiment, the manidipine is in the form of a pharmaceutically acceptable salt, preferably manidipine dihydrochloride.

In an embodiment, the sertraline is in the form of a pharmaceutically acceptable salt, preferably sertraline hydrochloride.

In an embodiment, the trifluoperazine is in the form of a pharmaceutically acceptable salt, preferably trifluoperazine dihydrochloride.

In an embodiment, the mycophenolate is in the form of a pharmaceutically acceptable salt, preferably mycophenolate mofetil.

There is also provided a pharmaceutical composition comprising a pharmaceutically acceptable excipient and one of the above combinations.

An “excipient,” as used herein, has its normal meaning in the art and is any ingredient that is not an active ingredient (drug) itself. Excipients include for example buffers, binders, lubricants, diluents, fillers, thickening agents, disintegrants, plasticizers, coatings, barrier layer formulations, stabilizing agent, release-delaying agents and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present composition is not limited in these respects. The carrier/excipient can be suitable, for example, for intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol, nebulizer) administration. Therapeutic compositions are prepared using standard methods known in the art by mixing the active ingredient having the desired degree of purity with one or more optional pharmaceutically acceptable carriers, excipients and/or stabilizers (see Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press).

In an embodiment, the compound or pharmaceutically acceptable salt thereof, or combination thereof, is/are formulated for oral administration. Formulations suitable for oral administration may include (a) liquid solutions, such as an effective amount of active agent(s)/composition(s) suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

In an embodiment, the compound or pharmaceutically acceptable salt thereof, or combination thereof, is/are formulated for parenteral administration (e.g., injection). Formulations for parenteral administration may, for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems include ethylenevinyl acetate copolymer particles, osmotic pumps, implantable infusion systems, and liposomes.

Formulations for inhalation may contain excipients, (e.g., lactose) or may be aqueous solutions containing, for example, polyoxyethylene-9-lauryl ether, glycocholate and deoxycholate, or may be oily solutions for administration in the form of nasal drops, or as a gel.

In an embodiment, the compound or pharmaceutically acceptable salt thereof, or combination thereof, is/are formulated for administration into the respiratory tract. In an embodiment, the compound or pharmaceutically acceptable salt thereof, or combination thereof, is/are formulated for pulmonary administration, e.g., in the form of an aerosol or spray. The compound or pharmaceutically acceptable salt thereof, or combination thereof, is/are may be administered using an inhalation device such as a metered-dose inhaler, dry powder inhaler or a nebulizer. Formulation for pulmonary administration typically include excipients such as sugars/polysaccharides, polymers, amino acids, viscosity modifiers, surfactants, propellants, lipids (e.g., to form liposomes), etc. Formulation for pulmonary administration may be in unit-dose or multidose presentations. In another aspect, the present disclosure provides an inhalation device comprising the compound or pharmaceutically acceptable salt thereof, or combination thereof. In another embodiment, the compound or pharmaceutically acceptable salt thereof, or combination thereof, is/are formulated for nasal administration, e.g., in the form of a nasal spray. In another embodiment, the compound or pharmaceutically acceptable salt thereof, or combination thereof, is/are formulated in the form of a mouth rinse.

The present disclosure provides a method for blocking the replication of a coronavirus such as SARS-CoV-2, in a coronavirus-infected cell, comprising contacting the cell with an effective amount of the compound, combination of compounds, or pharmaceutical composition described herein. The present disclosure also provides the use of the compound, combination of compounds, or pharmaceutical composition described herein for blocking the replication of a coronavirus such as SARS-CoV-2, in a coronavirus-infected cell. The present disclosure also provides the use of the compound, combination of compounds, or pharmaceutical composition described herein for the manufacture of a medicament for blocking the replication of a coronavirus such as SARS-CoV-2, in a coronavirus-infected cell. The present disclosure also provides the compound, combination of compounds, or pharmaceutical composition described herein for use in blocking the replication of a coronavirus such as SARS-CoV-2, in a coronavirus-infected cell.

The present disclosure provides a method for treating an infection by a coronavirus such as SARS-CoV-2, in a subject comprising administering to said subject an effective amount of the compound, combination of compounds, or pharmaceutical composition described herein. The present disclosure also provides the use of the compound, combination of compounds, or pharmaceutical composition described herein for treating an infection by a coronavirus such as SARS-CoV-2, in a subject. The present disclosure also provides the use of the compound, combination of compounds, or pharmaceutical composition described herein for the manufacture of a medicament for treating an infection by a coronavirus such as SARS-CoV-2, in a subject. The present disclosure also provides the compound, combination of compounds, or pharmaceutical composition described herein for use in treating a coronavirus such as SARS-CoV-2, in a subject.

The present disclosure provides a method for treating a viral disease caused by coronavirus such as COVID-19, in a subject comprising administering to said subject an effective amount of the compound, combination of compounds, or pharmaceutical composition described herein. The present disclosure also provides the use of the compound, combination of compounds, or pharmaceutical composition described herein for treating a viral disease caused by coronavirus such as COVID-19, in a subject. The present disclosure also provides the use of the compound, combination of compounds, or pharmaceutical composition described herein for the manufacture of a medicament for treating a viral disease caused by coronavirus such as COVID-19, in a subject. The present disclosure also provides the compound, combination of compounds, or pharmaceutical composition described herein for use in treating a viral disease caused by coronavirus such as COVID-19, in a subject.

The present disclosure provides a method for reducing the risk of developing a coronavirus-related disease such as COVID-19, or the severity of a coronavirus-related disease (e.g., COVID-19) in a subject, the method comprising administering to said subject an effective amount of the compound, combination of compounds, or pharmaceutical composition described herein. The present disclosure also provides the use of the compound, combination of compounds, or pharmaceutical composition described herein for reducing the risk of developing and/or the severity of a coronavirus-related disease such as COVID-19 in a subject. The present disclosure also provides the use of the compound, combination of compounds, or pharmaceutical composition described herein for the manufacture of a medicament for reducing the risk of developing a coronavirus-related disease such as COVID-19, or the severity of a coronavirus-related disease (e.g., COVID-19) in a subject. The present disclosure also provides the compound, combination of compounds, or pharmaceutical composition described herein for use in reducing the risk of developing a coronavirus-related disease such as COVID-19, or the severity of a coronavirus-related disease (e.g., COVID-19) in a subject.

For the prevention, treatment or reduction in the severity of a given disease or condition (viral disease such as COVID-19), the appropriate dosage of the compound or pharmaceutically acceptable salt thereof, or combination thereof described herein will depend on the type of disease or condition to be treated, the severity and course of the disease or condition, whether the compound or pharmaceutically acceptable salt thereof, or combination thereof is administered for preventive or therapeutic purposes, previous therapy, the patient's clinical history and response to the compound or pharmaceutically acceptable salt thereof, or combination thereof, and the discretion of the attending physician. The compound or pharmaceutically acceptable salt thereof, or combination thereof described herein may be suitably administered to the patient at one time or over a series of treatments. Preferably, it is desirable to determine the dose-response curve in vitro, and then in useful animal models prior to testing in humans. The present disclosure provides dosages for the compound or pharmaceutically acceptable salt thereof, or combination thereof described herein and compositions comprising same. For example, depending on the type and severity of the disease, about 1 μg/kg to to 1000 mg per kg (mg/kg) of body weight per day. Further, the effective dose may be 0.5 mg/kg, 1 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, and may increase by 25 mg/kg increments up to 1000 mg/kg, or may range between any two of the foregoing values. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

As used herein the term “treating” or “treatment” in reference to viral disease is meant to refer to a reduction/improvement in one or more symptoms or pathological features associated with said coronaviral disease (e.g., COVID-19), such as a reduction of the occurrence and/or severity of one or more symptoms. Non-limiting examples include a decrease in viral load, reduction of cough, fever, fatigue, shortness of breath, reduction/prevention of acute respiratory distress syndrome (ARDS), reduction/prevention of multi-organ failure, septic shock, blood clots, hospitalization, need for ICU involving intubation, etc.

Coronaviruses are large, roughly spherical, RNA viruses with bulbous surface projections that cause diseases in mammals and birds. In humans, these viruses cause respiratory tract infections that can range from mild to lethal. Mild illnesses include some cases of the common cold (which is also caused by other viruses, predominantly rhinoviruses), while more lethal varieties can cause severe acute respiratory syndrome (SARS), Middle East respiratory syndrome (MERS), and COVID-19.

In an embodiment, the methods and uses defined herein are for the prevention, treatment and/or management of infections by human Beta-Coronaviruses and/or associated diseases. Human Beta-Coronaviruses include OC43, HKU1, MERS-CoV, SARS-CoV and SARS-CoV-2. In a further embodiment, the methods and uses defined herein are for the prevention, treatment and/or management of infections by SARS-CoV-2 and associated disease (COVID-19). In an embodiment, the methods and uses defined herein are for the prevention, treatment and/or management of infections by the Wuhan original SARS-CoV-2 variant. In another embodiment, the methods and uses defined herein are for the prevention, treatment and/or management of infections by any variants of the Wuhan original SARS-CoV-2 variant. Examples of such variants include the B.1.1.7 (also known as VOC-202012/01 or alpha (α)), 501Y.V2 (also known as B.1.351 or beta (β)), P.1 (also known as B.1.1.28.1 or gamma (γ)), B.1.617.2 (also known as delta (δ)), or B.1.1.529 (omicron) variant, as well as other variants of concern (VOC) such as B.1.429, B.1.526, B.1.525, and A.23.1 (see, e.g., www.cdc.gov/coronavirus/2019-ncov/cases-updates/variant- surveillance/variant-info.html). In an embodiment, the methods and uses defined herein are for the prevention, treatment and/or management of infections by the SARS-CoV-2 Delta (δ) variant. In an embodiment, the methods and uses defined herein are for the prevention, treatment and/or management of infections by the SARS-CoV-2 Omicron variant, e.g., Omicron sublineage BA.1, BA.2 BA.3, BA.2.3.20, BA.2.75, BA.4.6, BA.5, and/or XBB. In an embodiment, the methods and uses defined herein are for the prevention, treatment and/or management of infections by any new SARS-CoV-2 variants.

In an embodiment, the combination of compounds or pharmaceutically acceptable salts thereof described herein exhibit a synergistic effect on the inhibition of coronavirus replication/infection. As used herein, a synergistic effect is achieved when the effect of the combined drugs is greater than the sum of the effect of each agent in the absence of the other. One potential advantage of combination therapy with a synergistic effect is that lower dosages (e.g., a suboptimal dose) of one or both of the drugs or therapies may be used in order to achieve high therapeutic activity with low toxicity. A further advantage of using the drugs in combination is that efficacy may be achieved in situations where either drug alone would not have an effect.

The compound or pharmaceutically acceptable salt thereof, or combination thereof described herein may be used alone or in combination with other prophylactic or therapeutic agents such as antivirals, anti-inflammatory agents, vaccines, immunotherapies, etc. The combination of active agents and/or compositions comprising same may be administered or co-administered (e.g., consecutively, simultaneously, at different times) in any conventional dosage form. Co-administration in the context of the present disclosure refers to the administration of more than one therapeutic throughout the course of a coordinated treatment to achieve an improved clinical outcome. Such co-administration may also be coextensive, that is, occurring during overlapping periods of time. For example, a first agent (e.g., the compound or combination of compounds described herein) may be administered to a patient before, concomitantly, before and after, or after a second active agent (e.g., an antiviral or anti-inflammatory agent) is administered. The agents may in an embodiment be combined/formulated in a single composition and thus administered at the same time.

In an embodiment, the compound or pharmaceutically acceptable salt thereof, or combination thereof described herein is for administration prior to development of the coronaviral disease (e.g., COVID-19). In another embodiment, the compound or pharmaceutically acceptable salt thereof, or combination thereof described herein is for administration after development of the coronaviral disease (e.g., COVID-19). In another embodiment, the compound or pharmaceutically acceptable salt thereof, or combination thereof described herein is for administration prior to and after development of the coronaviral disease (e.g., COVID-19).

In an embodiment, the subject or patient has a weakened immune system and a reduced ability to fight coronaviral infections such as SARS-CoV-2 infection. In another embodiment, the subject or patient is an immunosuppressed or immunocompromised subject or patient. Immunosuppression may be caused by certain diseases or conditions, such as AIDS, cancer, diabetes, malnutrition, and certain genetic disorders, or certain drugs or treatments such as anticancer drugs, radiation therapy, and stem cell or organ transplant. In an embodiment, the subject or patient is an elderly subject or patient, for example a subject or patient having 60 years old or more, 65 years old or more, 70 years old or more, 75 years old or more, or 80 years old or more, who typically develop a weaker immune response to vaccines and infections.

### Definitions

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted. In contrast, the phrase “consisting of” excludes any unspecified element, step, ingredient, or the like. The phrase “consisting essentially of” limits the scope to the specified materials or steps and those that do not materially affect the basic and novel characteristic(s) of the invention.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed.

No language in the specification should be construed as indicating any non-claimed element as essential to the practice of the invention.

Herein, the term “about” has its ordinary meaning. In embodiments, it may mean plus or minus 10% or plus or minus 5% of the numerical value qualified.

Unless otherwise defined, all technical and scientific terms used herein have the same meaning as commonly understood by one of ordinary skill in the art to which this invention belongs.

Other objects, advantages and features of the present invention will become more apparent upon reading of the following non-restrictive description of specific embodiments thereof, given by way of example only with reference to the accompanying drawings.

### Description of Illustrative Embodiments

The present invention is illustrated in further details by the following non-limiting examples.

### Example 1

### Abbreviations

BSL2-3: biosafety laboratory level 2 or 3.
CC₅₀: the Cytotoxic concentration of the extracts to cause death to 50% of viable cells.
EC₅₀: concentration of compound which inhibits pathogen at 50%.
MERS-CoV: Middle East respiratory syndrome.
MTT: methyl-thiazolyl-tetrazolium.
OC43: rHCoVs-OC43coronavirus.
Rluc: the Renilla luciferase.
SARS-CoV-2: Severe acute respiratory syndrome coronavirus 2.
Huh7.5: human hepatoma-derived HuH-7 cell line
Calu3: Human Lung Adenocarcinoma Cell Line.

### Materials and Methods (for Examples 1-3)

### Marketed and Approved Drug Library

The library was provided by NMX Research and Solutions Inc. (https://www.nmxresearch.com/). The library and individual compounds are available by Key Organics (https://www.keyorganics.net/services/bionet-products/fragment- libraries/).

### Cell Lines

HRT-18 cells were cultured in alpha-MEM medium (AMEM: Wisent) supplemented with 10% fetal bovine serum. Huh7.5, Calu3 and Vero E6 cells were cultured in Dulbecco's Modified Eagle Medium (DMEM, Wisent) supplemented with 10% fetal bovine serum (Wisent) and 1% of Minimum Essential Medium non-essential amino acid. All cell lines were incubated at 37° C. in atmosphere containing 5% CO₂.

### Viruses

rHCoVs-OC43 which express the Renilla luciferase (Rluc) reporter gene were obtained from Pierre Talbot and Wenjie Tan (Liang Shen et al., 2016). SARS-CoV-2 Vido virus (GISAID: EPI_ISL_413015) was obtained from VIDO-InterVac (Saskatchewan-Canada) and used for this study. SARS-CoV-2 LSPQ-B.1.1.7 Alpha/UK variant (GISAID: EPI_ISL_5873313) and SARS-CoV-2 LSPQ-B1-351 Beta/SA variant (GISAID: EPI_ISL_3458159) were obtained from LSPQ-INSPQ (Sainte-Anne-de-Bellevue-Canada) and used for this study.

Drug Tests in rHCoVs-OC43 Model

HRT-18 cells were plated in 10 cm cell culture dishes at 3*10⁶ cell/dishes and cultured over night at 37° C. Cells are infected or not by rHCoVs-OC43 during 4 h at 37° C. at MOI of 0.025. After virus infection, the cells are washed with PBS, trypsinised and seeded at 17,000 cells/mL in 96 well-plates. Each compound was added to the plate at a final concentration of concentrations 0.5, 1, 5, 10, 50, 100, 500, 1 000, 5 000 or 10 000 nM and an equal volume of DMSO was added in negative control wells. After 48 h of infection, cell viability and Rluc activity were measured with or without drugs to determine the best-drug candidates.

### Rluc Activity Assay

The Rluc analysis was performed in 96 well-plates. HRT-18 cells were lysed using Rluc lysis buffer (25 Mm glycyl-Glycine pH 7.8, 15 mM MgSO₄, 4 mM EGTA, 10% glycerol, 0.1% triton™ X-100). Rluc assays were made using coelenterazine diluted 700 times and quantification were made using TECAN.

### Drug Tests in SARS-CoV-2 Model

Huh7.5 cells were plated in 25 well-plate at 1×10⁵ cell/mL and cultured overnight at 37° C. Cells are infected or not by SARS-CoV-2 virus during 2 hours at 37° C. at MOI of 0.05. After virus infection, the cells were treated with drugs at concentrations 0.1, 1, 10, 100, 1 000, or 10 000 nM or an equal volume of DMSO was added in negative control. After 48 hours of treatment, the cells supernatant was collected and viral titration was determined. SARS-CoV-2 viral titration

Viral titrations were monitored by plaque assay method. In brief, 10-fold serial dilutions of supernatant containing an unknown amount of SARS-CoV-2 were absorbed on a monolayer of susceptible Vero E6 cells for 2 hours. After viral absorption, the supernatant was removed and a solid overlay Eagle's Minimal Essential Medium (EMEM, Wisent) containing 0.8% carboxymethylcellulose (SIGMA) was applied to the cells for 3 days. After fixation with 10% of formaldehyde, cells were wash and colored by 5% of crystal violet.

Cell Viability Assay (rHCoVs-OC43 and SARS-CoV-2)

Cell viability was monitored by a mitochondrial activity test using methyl-thiazolyl-tetrazolium (MTT, Sigma). After 48 hours of drug treatment, cells were treated with 1 mg/ml of MTT during 3 hours at 37° C. The supernatants were removed and the MTT crystals were dissolved using DMSO and a shaker. The absorbance was read with a Tecan Spark at 570 nm with reference at 650 nm.

Synergy Models for Drug Combination Studies (Adapted from Zheng et al., 2022)

For each drug combination, the degree of combination synergy/antagonism was determined. The synergy was quantified by comparing the observed drug combination response to the expected response. Considering the response of a drug to be measured as a percentage of inhibition that ranges from 0 to 100, a higher value indicates a better efficacy. Using a reference model that assumes there is no interaction between drugs. Experimental results available from SynergyFinder® (www.synergyfinderplus.org, Zheng et al., SynergyFinder Plus: Toward Better Interpretation and Annotation of Drug Combination Screening Datasets, Genomics Proteomics Bioinformatics 2022 Jan. 25; S1672-0229(22)00008-0. doi: 10.1016/j.gpb.2022.01.004.) were uploaded and all the analyses were run with the R package in the backend. For a combination that involves n drugs (n=2 in this study), the observed combination response is denoted as yc, whereas the observed monotherapy response of its constituent drugs is y_{i,i}=1, . . . n. Currently four major synergy reference models are used for synergy studies, HAS, BLISS, LOEWE and ZIP.

The HSA model, is one of the simplest reference models, which states that the expected combination effect is the maximum of the single drug responses at corresponding concentrations, that is, _{yHSA}=max(y₁ . . . ,y₂, . . . ,yₙ). The BLISS model, the expected response is derived from the probabilistic independence of the monotherapy effects, that is, _{yBLISS}=1− [symbol] i(1−yi). The LOEWE model, the expected response satisfies

$$\sum I ( \frac{xi}{{f}_{1}^{- 1} {(}_{yLOEWE} )} ) = 1 ,$$

where xᵢ is the dose of each constituent drug in the combination, and fᵢ⁻¹(_{yLOEWE}) is the inverse function of the dose-response curve. The ZIP model, the expected response satisfies _{yZIP}=1− [symbol] ᵢ(1−ŷᵢ), where ŷᵢ is the predicted dose response of the monotherapy by a monotonically increasing curve fitting model ŷᵢ=fᵢ(xᵢ)

### Overview of the Methods

Methods for identifying compounds that are active against coronaviruses replication were developed.
Step 1: Assembly and preparation of drugs stocks.
Step 2: Screen in coronavirus rHCoVs-OC43 assay for initial indicators of activity at reducing replication. The cell viability (CC₅₀) was also measured to determine the drugs toxicity.
Step 3: Identification and characterization of several compounds with antiviral activity against rHCoVs-OC43.
Step 4: Develop and validate a fast synergy screening method to promising drug combination using knew/published combination.
Step 5: Screening drugs in coronavirus rHCoVs-OC43 by fast synergy method.
Step 6: Implementing and validating coronavirus rHCoVs-OC43 “checkerboard” assays to screen and test synergy combination.
Step 7: Drug validations in coronavirus SARS-CoV-2 assays for EC₅₀.
Step 8: Synergy validation in SARS-CoV-2 using two different cell types: Huh7.5 and Calu3 cells.
   Then, singleton compounds that are active at reducing replication of coronavirus—as demonstrated with two representative Beta-coronaviruses, rHCoVs-OC43 and SARS-CoV-2, were identified. Finally, compounds that are active at reducing replication of coronavirus and at synergistically reducing replication of coronavirus SARS-CoV-2 as mixtures of compounds were identified.

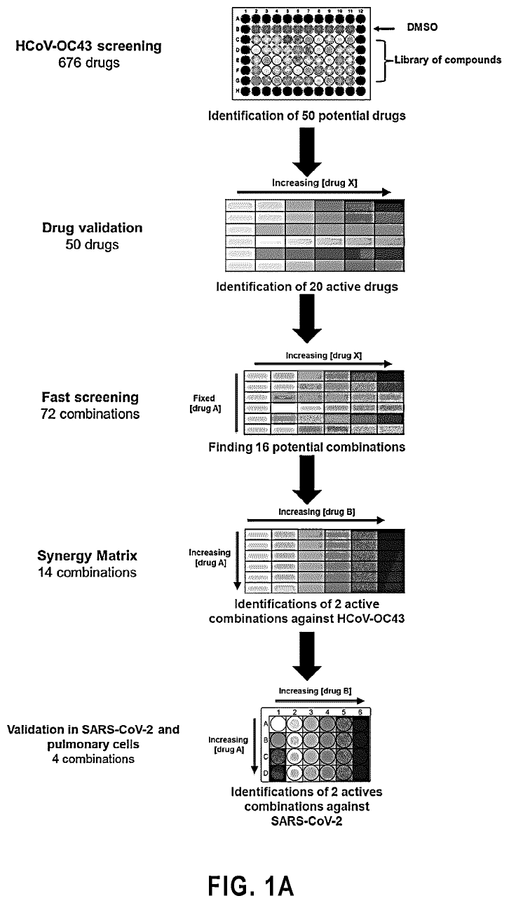

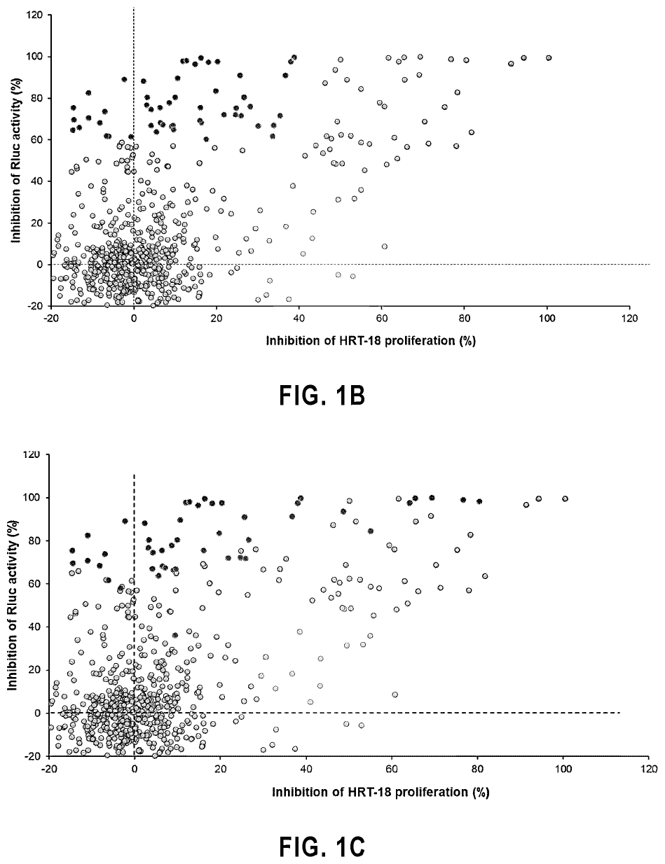

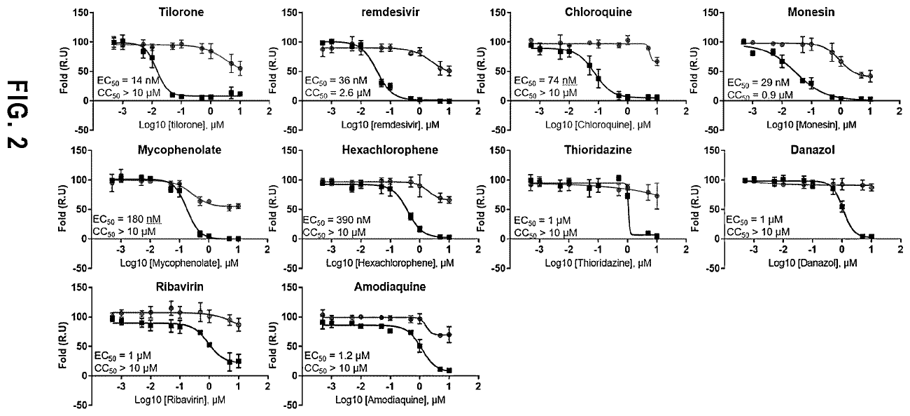

The methodology used is summarized in FIG. 1A. In this study, 676 compounds were screened against rHCoVs-OC43 virus, and 50 compounds were identified with potential antiviral activity. After calculation of EC₅₀ and CC₅₀, 20 compounds were characterized with good antiviral activity and low toxicity. A fast-screening approach has allowed to test 72 combinations and 14 potential combinations were identified. Matrix studies have permitted to identify 2 active combinations against rHCoVs-OC43. After validation of individual drugs activity against SARS-CoV-2, 2 active drugs were identified with promising activity against SARS-CoV-2.

FIGS. 1B and 1C show the screening results of the 676 drugs tested against rHCoVs-OC43. 65 drugs were identified with promising activity: 60-80/676 with good inhibition of Rluc activity-60 with Rluc inhibition >60% and grown inhibition <40%). These drugs are shown in FIG. 1B as full dots. Several parameters (Rluc inhibition, Growth inhibition, Rluc/growth inhibition ratio) were compared, and 50 drugs were chosen for further characterization. These drugs are shown in FIG. 1C as full dots. The antiviral activity was measured by the decrease of Rluc reporter signal (EC₅₀) and the cellular viability by MTT test (CC₅₀).

The methodology is further described below as Steps 1-8.

### Step 1: Assembly and Preparation of Drugs Stocks

Step 1 involved securing pure drugs for testing purposes. For the initial part of this research, 676 drugs were purchased from the commercial chemical supplier Key Organics. The 676 compounds are classified as marketed and approved drugs for indications other than coronaviruses. The appropriate amounts of powder-form of the drugs were dissolved in dimethyl sulphoxide-d6 (DMSO) to achieve a nominal concentration of 50 mM.

### Step 2: Screen in Coronavirus rHCoVs-OC43 Assay for Initial Indicators of Activity at Reducing Replication

Using the rOC43-nsDel-Rluc reporter virus, the potential antiviral activity of the drugs was determined. Briefly HRT-18 cells were infected with rHCoVs-OC43 virus at MOI of 0.025, during 4 hours, and treated for 48 hours with 10 μM of each drug. The antiviral activity was measured by the decrease of the Renilla Luciferase (Rluc) assay and the cellular viability by a methyl-thiazolyl-tetrazolium (MTT) test.

For this, 676 drugs were screened against rHCoVs-OC43 and the FIG. 1B displays the results. Where it was noted that 60-80 of 676 compounds exhibited more than 60% inhibition of replication in the Rluc activity and less than 40% in growth inhibition MTT assay, black dots (HRT-18 proliferation assay). The selection criteria were refined, and the 50 best compounds were selected (black dots) based on the most promising Rluc/growth inhibition ratio and chemical structures of interest (see FIG. 1C).

### Step 3: Identification and Characterization of Several Compounds with Antiviral Activity Against rHCoVs-OC43

Once validated, the 676 drugs were screened as singleton compounds in a coronavirus rHCoVs-OC43 assay as described in the Materials and Methods section of this application. Briefly, HRT-18 cells were infected with rHCoVs-OC43 virus at MOI of 0.025, during 4 hours, and treated for 48 hours with a complete range of drugs (0.5, 1, 5, 10, 50, 100, 500, 1 000, 5 000 or 10 000 nM). The antiviral activity was measured by the decrease of RLuc assay and the cellular viability by MTT test. The half-maximal inhibitory concentration was determined to the antiviral activity as EC₅₀ and to the cellular viability as CC_{50.}

FIG. 2 shows the resultant EC₅₀ (squares) and CC₅₀ (dots) curves of the top 10 drugs. The impressive activities of these drugs can be better evaluated based on the squares and accompanying curves from which EC₅₀ values for inhibition of rHCoVs-OC43 replication can be derived. For comparison purposes, one can derive a relative toxicity measurement (CC₅₀) based on the dots accompanying and curves.

The chemical structures of the 10 best drugs against rHCoVs-OC43 are shown below.

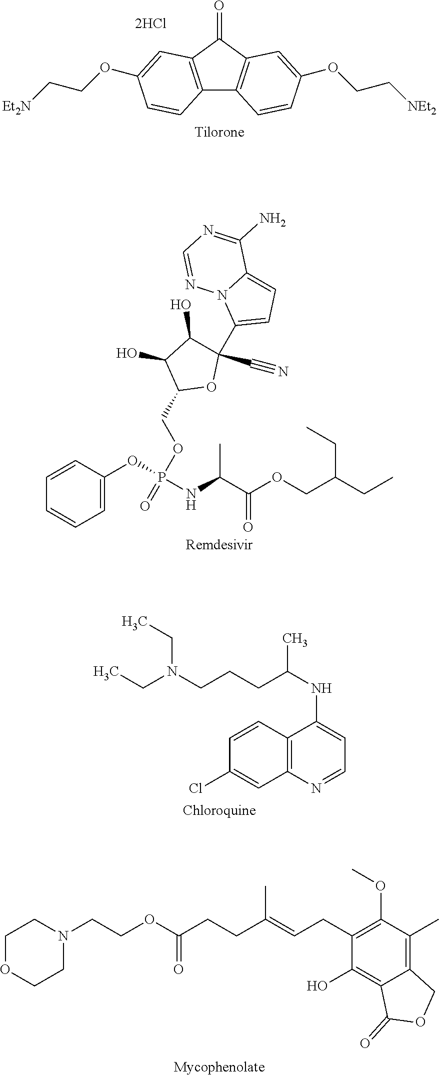

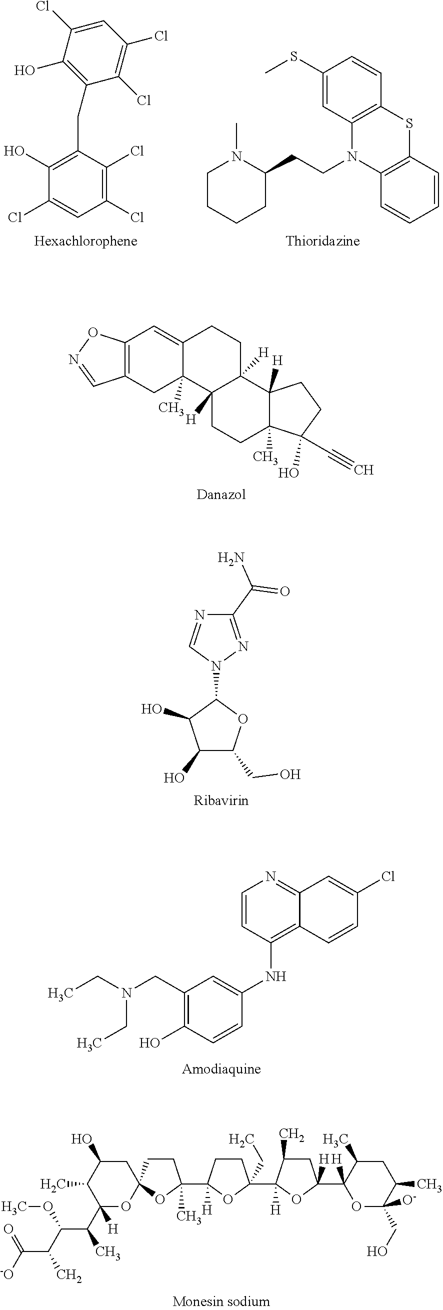

The table below shows the EC₅₀ and CC₅₀ values for these 50 drugs.

| Drugs | EC₅₀ (μM) | CC₅₀ (μM) |
|---|---|---|
| Remdesivir | 0.034 | 2.5 |
| Fingolimod hydrochloride | 3.9 | 10 |
| Thioridazine hydrochloride | 1.0 | >10 |
| Mycophenolate mofetil | 0.180 | >10 |
| Amlodipine benzenesulphonate | 5.2 | >10 |
| Sertraline hydrochloride | 2.3 | >10 |
| Vortioxetine | 2.4 | >10 |
| Manidipine dihydrochloride | 4.5 | >10 |
| Vortioxetine hydrobromide | 2.4 | >10 |
| Trifluoperazine dihydrochloride | 2.9 | >10 |
| Amiodarone hydrochloride | 2.3 | >10 |
| Nelfinavir mesylate | 2.6 | >10 |
| Risperidone | >10 | >10 |
| Alfacalcidol | 1.7 | >10 |
| Danazol | 1.0 | >10 |
| Tacrolimus hydrate | 3.3 | >10 |
| Toremifene Citrate | 2.6 | >10 |
| chlorpromazine | 5.1 | >10 |
| Nisoldipine | 2.35 | >10 |
| Haloperidol decanoate | >10 | >10 |
| Duloxetine hydrochloride | 4.8 | >10 |
| Dapoxetine hydrochloride | 4.7 | >10 |
| Clomipramine hydrochloride | 3.8 | >10 |
| Ebastine | >10 | >10 |
| Tamoxifen | 3.4 | >10 |
| Tioconazole | 4.1 | >10 |
| Nitazoxanide | 1.33 | >10 |
| Rilpivirine | >10 | 10 |
| Amodiaquine | 1.2 | >10 |
| Doxazosin mesylate | 3.2 | >10 |
| Clobetasone butyrate | 8.5 | >10 |
| Chlorprothixene | 7.5 | >10 |
| Orlistat | 7.3 | >10 |
| Rafoxanide | 6.9 | >10 |
| Tilorone dihydrochloride | 0.014 | >10 |
| Benproperine phosphate | 5.6 | >10 |
| Dipyridamole | 4.1 | >10 |
| Chloroquine | 0.066 | >10 |
| Enzastaurin hydrochloride | 2.8 | >10 |
| Pimanavserin | 6.0 | >10 |
| Ribavirin | 1.0 | >10 |
| Disulfiram | 0.180 | 0.230 |
| Ouabain | 0.016 | 0.028 |
| Digoxin | 0.028 | 0.075 |
| Hexachlorophene | 0.390 | >10 |
| Clemastine fumarate | 5.0 | >10 |
| Clomiphene citrate | 4.5 | >10 |
| Digitoxin | 0.021 | 0.080 |
| Monesin sodium sulfate | 0.029 | 0.900 |
| Clofazimine | 1.8 | >10 |

### Example 2

### Step 4: Develop and Validate a Fast Synergy Screening Method to Promising Drug Combination Using Knew/Published Combination

Having determined the top candidate compounds, the next goal was to determine if combinations of the drugs would be synergistic or antagonistic with regards to antiviral activity. For Step 4, coronavirus rHCoVs-OC43 “checkerboard” assays were implemented and validated as a means to rapidly screen and test for synergy. For throughput reasons, an assay involving the representative beta-coronavirus rHCoVs-OC43 has many advantages over assays involving SARS-CoV-2. One big advantage is that an assay involving rHCoVs-OC43 can be performed under bio-safety level 2 (BSL2) conditions (using a bio-safety hood) which is amenable to higher and faster throughput, as compared to the much slower throughput required for an assay involving SARS-CoV-2 which must be performed in a highly regulated BSL3 laboratory.

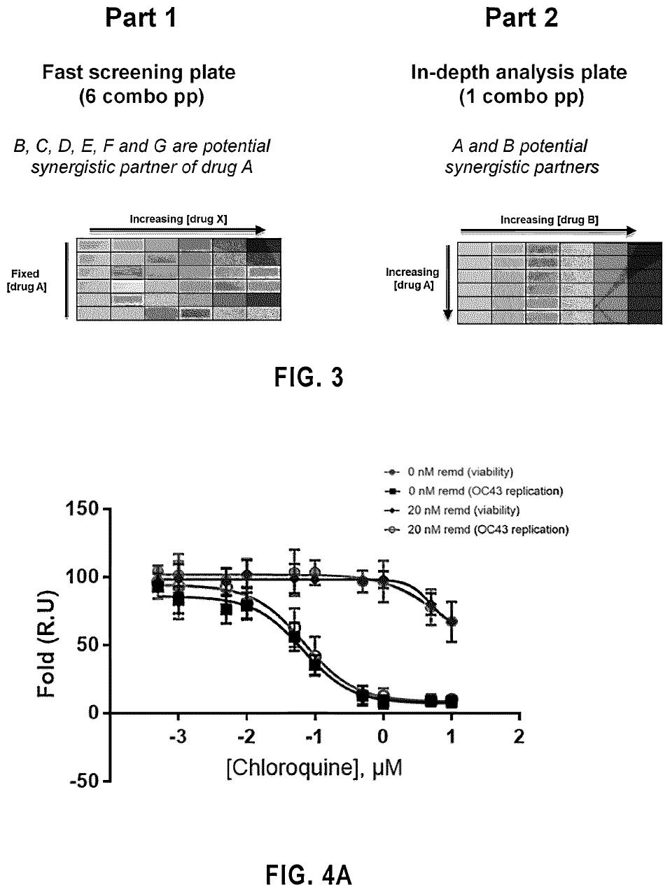

FIG. 3 shows diagrams of the two-part customized process that was implemented. Part 1 was a method to rapidly screen a single compound (drug A) at a fixed concentration against six compounds (drugs B-G) at increasing concentrations. The purpose was to qualitatively evaluate if any of the six drugs produced signs of potential synergic activities. If potential synergistic effects were noted, the more in-depth screen and analysis was performed in Part 2 where drug A plated at increasing concentrations (vertical axis) and drug B was plated at increasing concentrations along the horizontal axis. Data from Part 1 and 2 were then used as input into the software Synergyfinder.

Overall, the software analyzed the input experimental values and compared them using mathematical equations (i.e., ZIP, Bliss, Loewe and HSA models). The experimental dose-response surface data that delineates combination effects in concentration space, was first read by the software as a matrix of percentage of the control value across concentrations. Single agent effects were extracted from this data and fitted with a dose response curve. Based on the two single agent dose response curves, a model-based combination dose-response surface was derived. This surface provided a ‘reference’ dose-response surface for a non-synergistic (additive/independent) combination, whose characteristics were determined by the selected model (i.e., ZIP, Bliss, Loewe and HSA models). The experimental combination dose response surface was then compared to the model-generated one, resulting in a synergy distribution in concentration space.

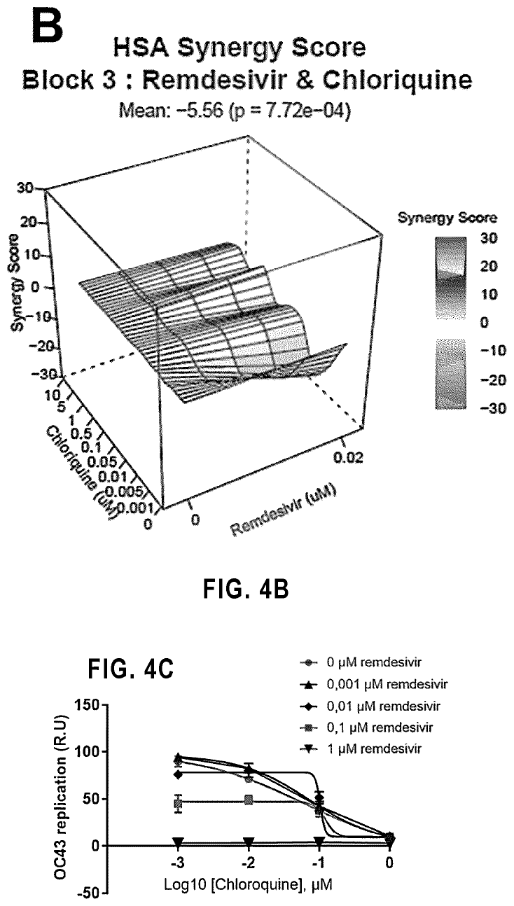

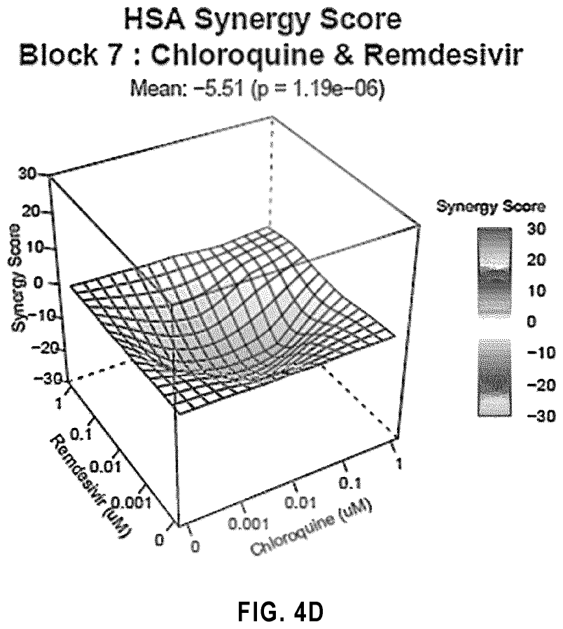

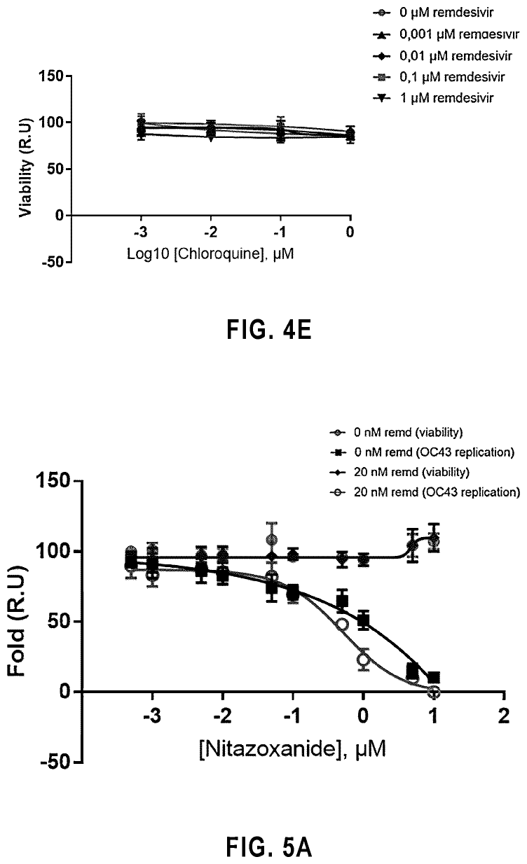

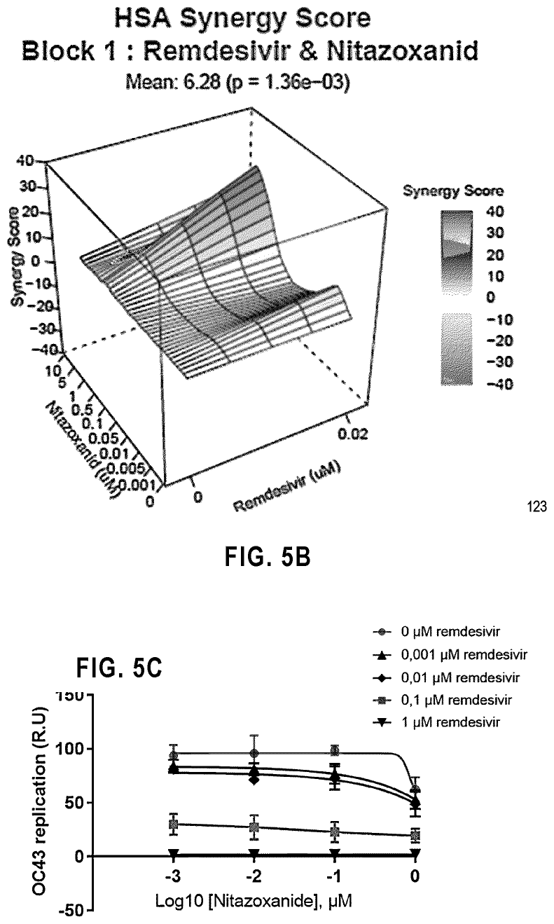

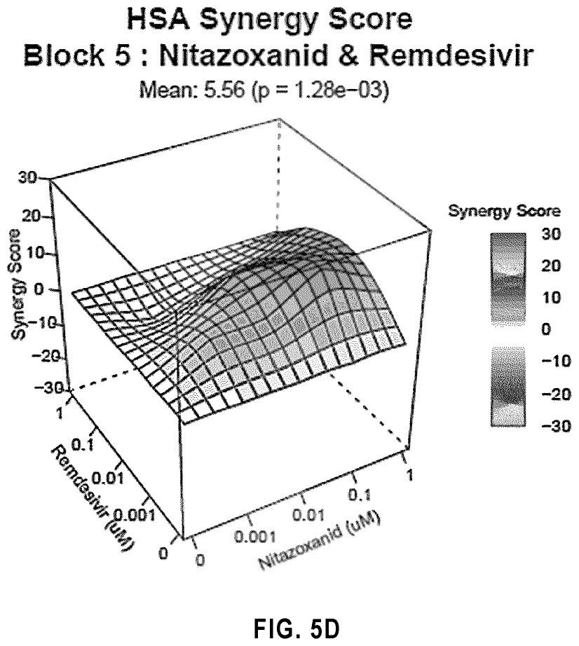

The different combinations were classified according to the simplest parameter: HSA. The other parameters were also measured and considered in the analysis. The dose-response data and analyses can be visualized as surface-matrix format (FIG. 4B-D, 5B-D).

### Antagonistic Combination

The software was put to a practical evaluation in partially controlled experiments. Based on literature reports, it was known that the combination of remdesivir and chloroquine exhibited antagonistic effects on SARS-CoV-2 viral replication. Assuming that this may also be the case for rHCoVs-OC43, experiments were performed on rHCoVs-OC43 and data analyzed using the Synergyfinder software and visualizable in FIG. 4.

Using this antagonist combination, the screening model was validated (FIG. 4A-B). Two curves are represented on FIG. 4A, and the viral replication measured in the presence of a full range of chloroquine with (empty circles) or without 20 nM of remdesivir (black squares) can be compared. The inhibitory effect of the combination is weaker than when chloroquine is used alone. FIG. 4B makes it possible to determine the synergistic effect or not of the combination.

The study of FIG. 4C shows that the different viral replication inhibition curves overlap. FIG. 4D, meanwhile, shows an example of entirely negative synergistic values.

Finally, FIGS. 4A and 4E, in which the cell viability is studied, do not show a greater toxic effect during the studies of combinations (chloroquine and remdesivir).

### Synergistic Combination

The software was put to another practical evaluation in partially controlled experiments. Based on literature reports, it was known that the combination of remdesivir and nitazoxanide exhibited synergic effects on SARS-CoV-2 viral replication. Assuming that this may also be the case for rHCoVs-OC43, experiments were performed on rHCoVs-OC43 and data analyzed using the Synergyfinder software and visualizable in FIG. 5.

Using this synergistic combination, the screening model was validated (FIG. 5AB). Two curves are represented on FIG. 5A, and the viral replication measured in the presence of a full range of nitazoxanide with (circles) or without 20 nM of remdesivir (squares) can be compared. The inhibitory effect of the combination is better than when nitazoxanide is used alone. FIG. 5B makes it possible to determine the synergistic effect or not of the combination.

The study of FIG. 5C shows that the different viral replication inhibition curves. A better inhibition was obtained when the remdesivir concentration was increased. FIG. 5D, meanwhile, shows an example of entirely positive synergistic values.

Finally, FIGS. 5A and 5E, in which the cell viability is studied, do not show a greater toxic effect during the studies of combinations (nitazoxanide and remdesivir).

### Step 5: Screening Drugs in Coronavirus rHCoVs-OC43 by Fast Synergy Method

The screening of drugs in coronavirus OC43 assays was used to select those that exhibited synergy effects. The table below summarizes results observed for remdesivir (A), tilorone (B) or mycophenolate (C) in combination with other drugs. In order to determine if combinations will be promising, several parameters were measured and gathered in the table below: the synergy results (ZIP, Loewe, HAS and Bliss methods), the EC₅₀ and CC₅₀ results for drug 2 alone, and the CC₅₀ synergy results. The drugs with strong synergistic potential, without affecting cell viability were selected. Thus, combinations with remdesivir (A) or tilorone (B) are more promising than those obtained with mycophenolate (C).

TABLE
Screening with complete range of drugs and 20 nM of remdesivir (A). 10 nM of tilorone (B). or 40 nM of
mycophenolate (C).

| | | | | | | | | | CC₅₀ | |
|---|---|---|---|---|---|---|---|---|---|---|
| | | | | | | | IC₅₀ | CC₅₀ | synergy | Viability |
| A | Drug 1 | Drug 2 | ZIP | Loewe | HSA | Bliss | (μM) | (μM) | (μM) | affected |
| | Remdesivir | Amiodarone | −5.3 | −3.11 | −4.47 | −9.3 | 2.3 | >10 | >10 | no |
| | | Amlodipine | −0.37 | 4.23 | 5.6 | 1.14 | 5.2 | >10 | >10 | no |
| | | Amodiaquine | −6.8 | −8.75 | −8.88 | −10.86 | 1.2 | >10 | >10 | no |
| | | Chloriquine | −4.37 | −4.69 | −5.56 | −8.47 | 0.066 | >10 | >10 | no |
| | | Clemastine | −7.72 | −13.73 | −14.53 | −15.9 | 5 | >10 | >10 | no |
| | | Clofazimine | −6.18 | −6.06 | −5.9 | −10.98 | 1.8 | >10 | >10 | no |
| | | Clomiphene | 0.61 | −9.5 | −9.62 | −4.58 | 4.5 | >10 | >10 | no |
| | | Digitoxin | −1.85 | −9.75 | −10.16 | −6.96 | 0.021 | 0.08 | 0.029 | yes |
| | | Digoxin | 0.62 | 5.31 | 5.57 | −0.38 | 0.028 | 0.075 | 0.075 | no |
| | | Fingolimod | −13.81 | −19.24 | −19.02 | −19.63 | 3.9 | >10 | >10 | no |
| | | Hexachlorophene | 6.37 | 7.9 | 7.56 | 6.07 | 0.39 | >10 | >10 | no |
| | | Monesin sodium | −56.95 | −54.42 | −53.94 | −57.43 | 0.029 | 0.9 | 0.85 | yes |
| | | Mycophenolate | 1.02 | 2.53 | 2.58 | 1.25 | 0.18 | >10 | >10 | no |
| | | Nelfinavir | 0.15 | 4.29 | 4.27 | 0.02 | 2.6 | >10 | >10 | no |
| | | Nitazoxanide | 1.36 | 6.62 | 6.28 | 0.79 | 1.33 | >10 | >10 | no |
| | | Oubain | −0.96 | 3.86 | 4.23 | −0.04 | 0.016 | 0.028 | 0.044 | no |
| | | Ribavirin | −9.95 | −10.83 | −11.08 | −17.09 | 1 | >10 | >10 | no |
| | | Thioridazine | −5.78 | −3.8 | −3.72 | −8.62 | 1 | >10 | >10 | no |
| | | Toremifene | −2.15 | 2.18 | 2.39 | −2.6 | 2.6 | >10 | >10 | no |
| | | Vortioxetine | −3.89 | −4.62 | −4.29 | −7.23 | 2.4 | >10 | >10 | no |
| | | | | | | | | | CC₅₀ | |
| | | | | | | | IC₅₀ | CC₅₀ | synergy | Viability |
| B | Drug 1 | Drug 2 | ZIP | Loewe | HSA | Bliss | (μM) | (μM) | (μM) | affected |
| | Tilorone | Alfacalcidol | 13.05 | 16.85 | 16.08 | 13.66 | 1.7 | >10 | >10 | no |
| | | Amiodarone | −3.97 | 1.24 | 1.02 | −6.88 | 2.3 | >10 | >10 | no |
| | | Amlodipinebenzene | −8.12 | −8.18 | −8.23 | −13.89 | 5.2 | >10 | >10 | no |
| | | Amodiaquine | 0.55 | 2.18 | 1.16 | −1.28 | 1.2 | >10 | >10 | no |
| | | Clofazimine | 4.18 | 4.7 | 3.43 | 4.12 | 1.8 | >10 | >10 | no |
| | | Danazol | 10.94 | 12.54 | 12.2 | 12.66 | 1 | >10 | >10 | no |
| | | Fingolimod | −3.47 | −9.91 | −9.59 | −6.64 | 3.9 | 10 | 6.9 | yes |
| | | Manidipine | 3.93 | 5.5 | 7.34 | 4.73 | 4.5 | >10 | >10 | no |
| | | Nelfinavir | 6 | 9.74 | 9.85 | 6.72 | 2.6 | >10 | >10 | no |
| | | Remdesivir | 2.98 | 4.59 | 4.69 | 2.84 | 0.034 | 2.5 | 2.5 | no |
| | | Ribavirin | 7.74 | −13.17 | −13.12 | −13.11 | 1 | >10 | >10 | no |
| | | Sertraline | 9.14 | 8.63 | 7.48 | 8.7 | 2.4 | >10 | >10 | no |
| | | Thioridazine | −3.3 | −1.61 | −1.75 | −5.19 | 1 | >10 | 5.3 | yes |
| | | Trifluoperazine | 7.08 | 7.33 | 7.77 | 7.77 | 2.9 | >10 | 8.9 | yes |
| | | Vortioxetine | 0.76 | −0.62 | −0.74 | −2.98 | 2.4 | >10 | >10 | no |
| | | | | | | | | | CC₅₀ | |
| | | | | | | | IC₅₀ | CC₅₀ | synergy | Viability |
| C | Drug 1 | Drug 2 | ZIP | Loewe | HSA | Bliss | (μM) | (μM) | (μM) | affected |
| | Mycophenolate | Alfacalcidol | 6.99 | 5.05 | 4.5 | 5.87 | 1.7 | >10 | >10 | no |
| | | Amiodarone | 6.25 | 6.17 | 5.49 | 5.64 | 2.3 | >10 | >10 | no |
| | | Amlodipine | 0.39 | −9.61 | 7.08 | −2.23 | 5.2 | >10 | >10 | no |
| | | Amodiaquine | 5.29 | 2.14 | 1.12 | 3.29 | 1.2 | >10 | >10 | no |
| | | Clofazimine | −4.41 | −2.69 | −2.72 | −7.06 | 1.8 | >10 | >10 | no |
| | | Danazol | 13.2 | 6.18 | 6.44 | 13.57 | 1 | >10 | >10 | no |
| | | Fingolimod | 3.71 | 0.33 | 2.15 | 5.31 | 3.9 | 10 | 6.6 | yes |
| | | Manidipine | 20.4 | 12.99 | 14.79 | 21.51 | 4.5 | >10 | 10 | yes |
| | | Nelfinavir | 16.31 | 18.06 | 17.72 | 16.94 | 2.6 | >10 | 9.12 | yes |
| | | Remdesivir | 6.81 | 8.5 | 8.49 | 7.1 | 0.034 | 2.5 | 2.26 | yes |
| | | Ribavirin | −5.96 | −8.84 | −9.16 | −10.94 | 1 | >10 | >10 | no |
| | | Sertraline | 5.41 | 5.56 | 5.5 | 4.89 | 2.4 | >10 | >10 | no |
| | | Thioridazine | 1.61 | −2.2 | −2.51 | 0.53 | 1 | >10 | >10 | no |
| | | Trifluoperazine | 4.51 | 3.66 | 4.03 | 3.93 | 2.9 | >10 | 10 | yes |
| | | Vortioxetine | −2.93 | −7.19 | −7.25 | −9.2 | 2.4 | >10 | 10 | yes |

### Step 6: Implementing and Validating Coronavirus rHCoVs-OC43 “Checkerboard” Assays to Screen and Test Synergy Combination

Further studies were performed with alternate combinations of drugs and the results are summarized in the two tables below. To find synergistic combination, 16 combinations have been tested by matrix method with remdesivir, mycophenolate or tilorone on the one hand and with more than 10 other drugs on the other hand.

TABLE
Identification of several synergistic drug combinations in
rHCoVs-OC43. Table with synergy matrix tested as 5 × 5 format (A).

| Drug 1 | Drug 2 | ZIP | Loewe | HSA | Bliss |
|---|---|---|---|---|---|
| Nitazoxanide | Remdesivir | 3.01 | 4.31 | 5.56 | 2.51 |
| Chloroquine | | −8.71 | −6.86 | −5.51 | −11.24 |
| Chlofazimine | | −8.68 | −3.41 | −3.57 | −10.34 |
| Nelfinavir | | −4.73 | −3.15 | −3.07 | −4.34 |
| Mycophenolate | | 0.45 | 1.63 | 2.95 | 1.1 |
| Amodiaquine | | −5.9 | −3.24 | −2.71 | −5.61 |
| Vortioxetine | | −2.1 | 0.54 | 0.64 | −3.48 |
| Toremifene | | −0.66 | 0.45 | 0.03 | −0.61 |

This modified and customized checkerboard method involved an increase in the number of concentration points. Therefore, the first tests were done with 5 concentrations of drug 1 (remdesivir) and 5 concentrations of drug 2 (see the above table), and gave promising results for combinations of:
nitazoxanide and remdesivir, and
mycophenolate and remdesivir.

The range of concentration being too small, several points were added. Then, the best results were tested with 5 concentrations of drug 2 (either remdesivir, tilorone, mycophenolate) and 10 concentrations of drug 1 (see the table below).

TABLE
Identification of several synergistic drug combinations in
rHCoVs-OC43. Table with synergy matrix tested as 5 × 10 format (B).

| Drug 1 | Drug 2 | ZIP | Loewe | HSA | Bliss |
|---|---|---|---|---|---|
| Nitazoxanide | Remdesivir | 3.47 | 2.91 | 3.98 | 2.53 |
| Tilorone | | −0.13 | 0.82 | 2.59 | −0.4 |
| Mycophenolate | | 1.17 | 0.24 | 2.06 | 1.24 |
| Toremifene | | −1.31 | 0.25 | 1.58 | −0.76 |
| Nelfinavir | Mycophenolate | −0.62 | −6.94 | −7.61 | −2.61 |
| Trifluoperazine | | 11.12 | −3.47 | 1.91 | 13.42 |
| Nelfinavir | Tilorone | 6.76 | 6.14 | 7.62 | 5.95 |
| Trifluoperazine | | 8.24 | 4.65 | 6.38 | 7.22 |

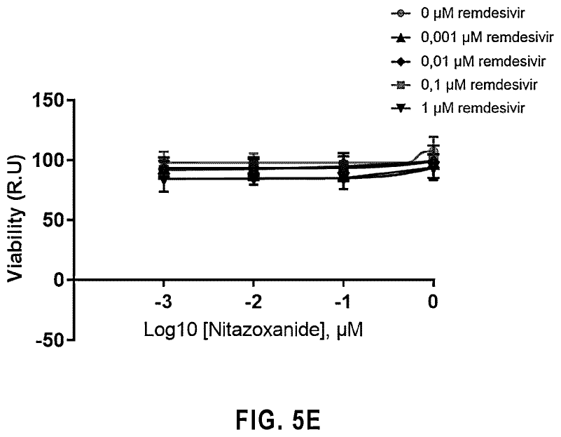

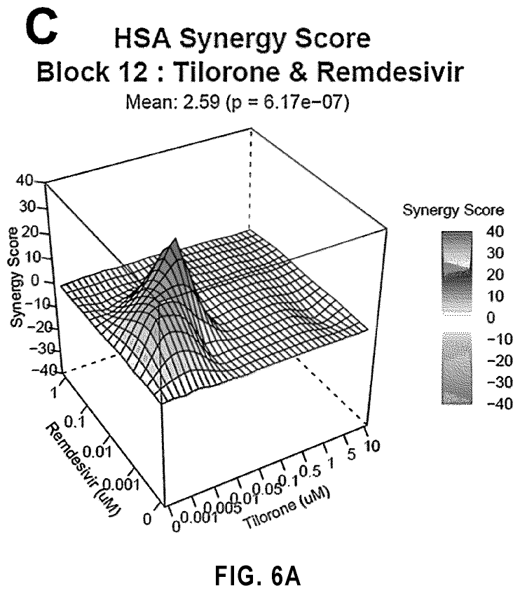

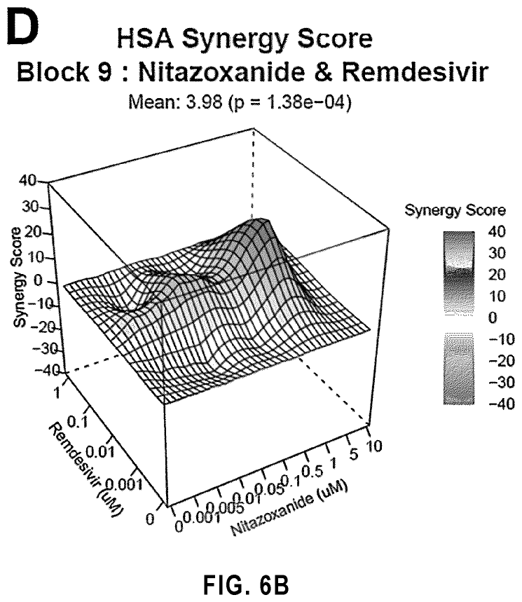

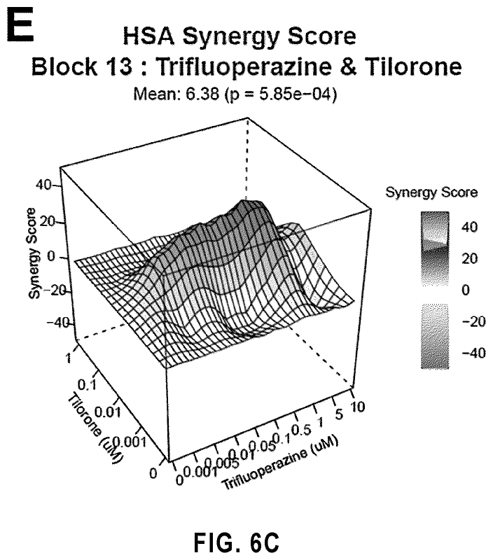

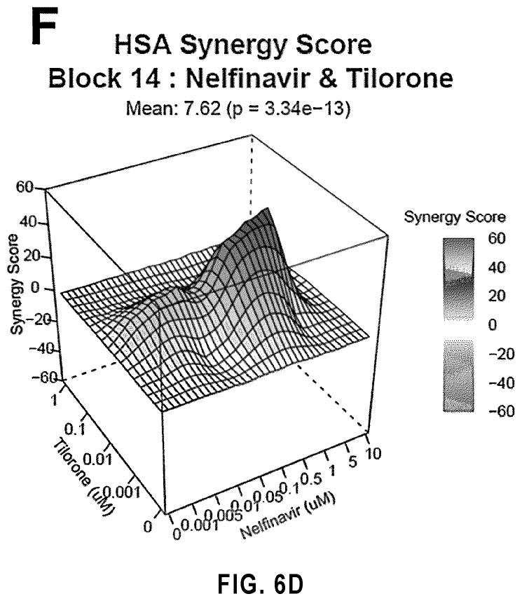

The best results were obtained with the following combinations:
tilorone-remdesivir (FIG. 6A),
nitazoxanide-remdesivir (FIG. 6B),
trifluoperazine-tilorone (FIG. 6C) and
nelfinavir-tilorone (FIG. 6D).

For each combination tested, it was assessed whether the combined drugs were more toxic to cells than the drugs used alone.

The mycophenolate/trifluoperazine and mycophenolate/nelfinavir combinations presented the higher toxicity and were eliminated despite interesting synergy scores obtained for the mycophenolate/trifluoperazine combination.

These results provide evidence that a drug combination involving tilorone with nelfinavir or remdesivir could serve to reduce coronavirus replication and serve as medications for diseases related to coronaviruses.

### Step 7: Drug Validations in Coronavirus SARS-CoV-2 Assays for EC₅₀ and CC₅₀ using two different cell types: Huh7.5 and Calu3.

The first step to determine if a combination is promising against SARS-CoV-2 was to test each drug alone and the results are summarized in FIG. 7. The efficacy against SARS-CoV-2 was determine in two different cell types, the common hepatocarcinoma cell line Huh7.5 usually used in drug discovery and the pulmonary cell line Calu3 more specific for pulmonary disease. The efficacy was determined by measuring the viral levels of SARS-CoV-2 by viral titration in VeroE6 cells.

It was decided to add the known SARS-CoV-2 drugs molnupinavir (Merck) and Paxlovid (Pfizer) to the candidate molecules. Since these drugs have been identified and published as inhibitors of SARS-CoV-2, no test has been done with the rHCoVs-OC43 virus.

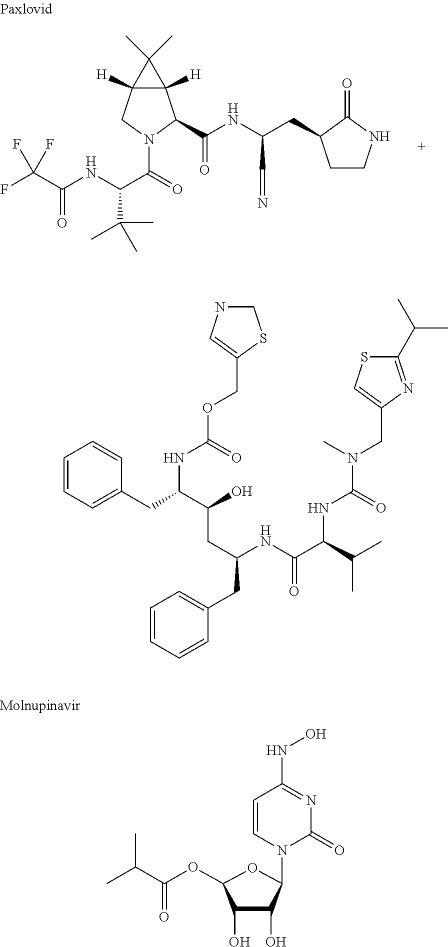

Most of the candidate molecules showed an inhibitory effect on viral replication (FIG. 7) with a fairly low toxicity in both cell lines (see tables below). Only the most promising molecules or those studied in synergy were tested in Calu3 cells. The promising molecule trifluoperazine did not show any significant inhibition of viral replication. The cell viability was measured in for each molecule via the MTT test, and no molecule shows high toxicity.

The viral concentration was measured by plate assay method in Vero E6 cells and the cellular viability by MTT test (B-C). EC₅₀ and CC₅₀ were measured in Huh7.5 (B) and Calu3 (C) and the results are summarized in the tables below.

| B | | | C | | |
|---|---|---|---|---|---|
| | SARS- | SARS- | | SARS- | SARS- |
| | CoV-2 | CoV-2 | | CoV-2 | CoV-2 |
| | EC₅₀ | CC₅₀ | | EC₅₀ | CC₅₀ |
| Drugs | (μM) | (μM) | Drugs | (μM) | (μM) |
| Tilorone | 0.5 | >10 | Tilorone | 0.070 | >10 |
| Nelfinavir | 1.7 | >10 | Nelfinavir | 0.11 | >10 |
| Remdesivir | ≤0.0010 | >10 | Remdesivir | 0.004 | >10 |
| Trifluoperazine | >10 | >10 | Molnupinavir | 0.035 | >10 |
| Mycophenolate | 1.18 | 9.1 | Paxlovid | 0.013 | >10 |
| Danazol | 0.83 | >10 | | | |
| Nitazoxanide | 0.54 | >10 | | | |
| Molnupinavir | 0.084 | >10 | | | |
| Paxlovid | 0.054 | >10 | | | |

### Step 8: Synergy Validation in SARS-CoV-2 Using Two Different Cell Types: Huh7.5 and Calu3 cells

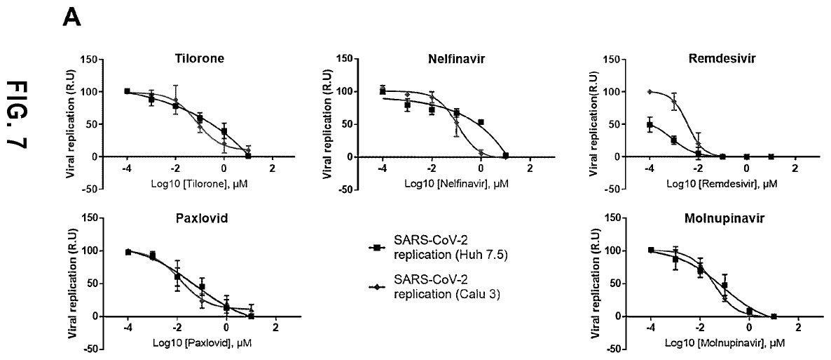

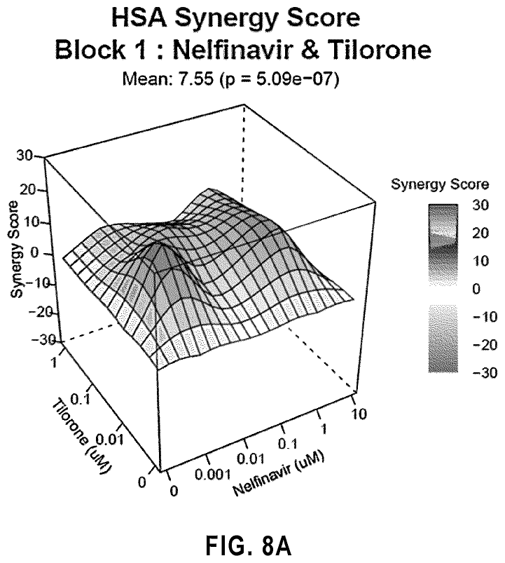

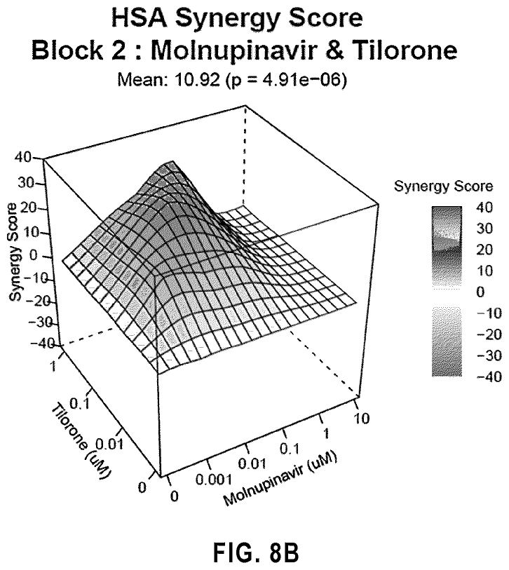

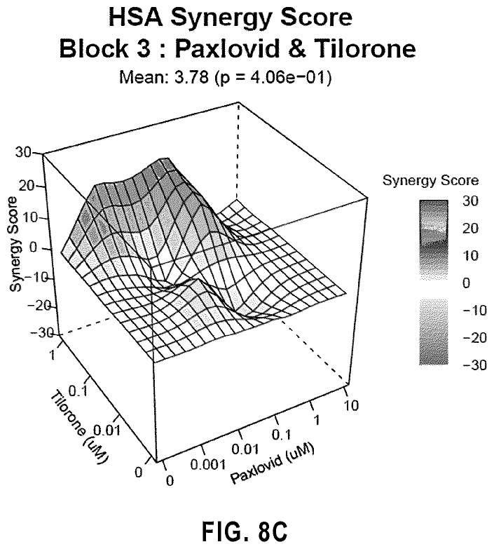

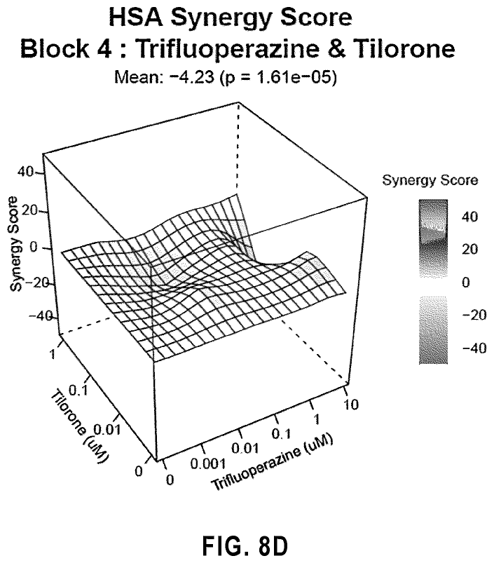

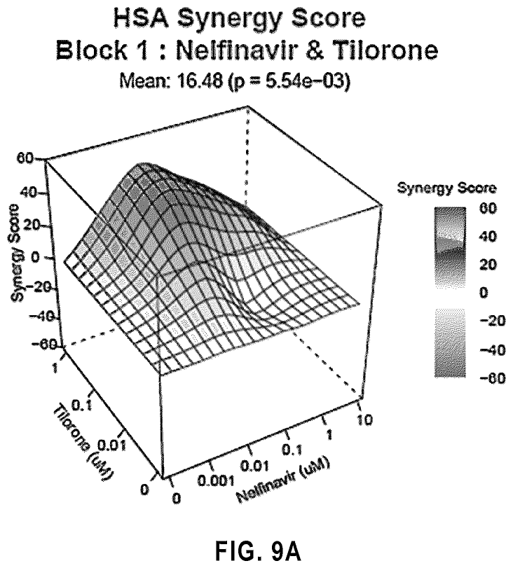

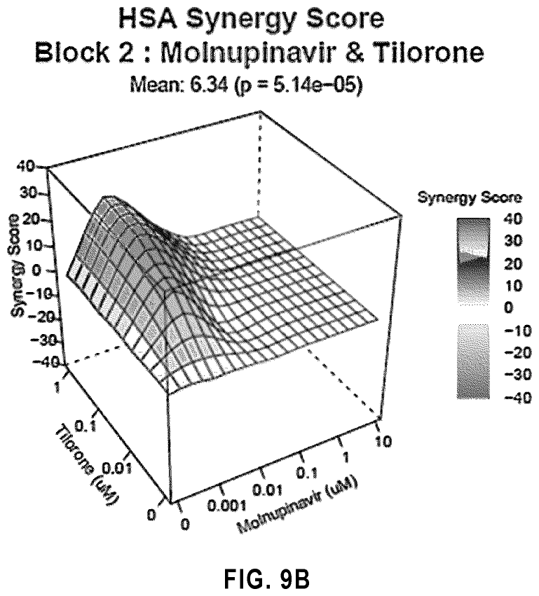

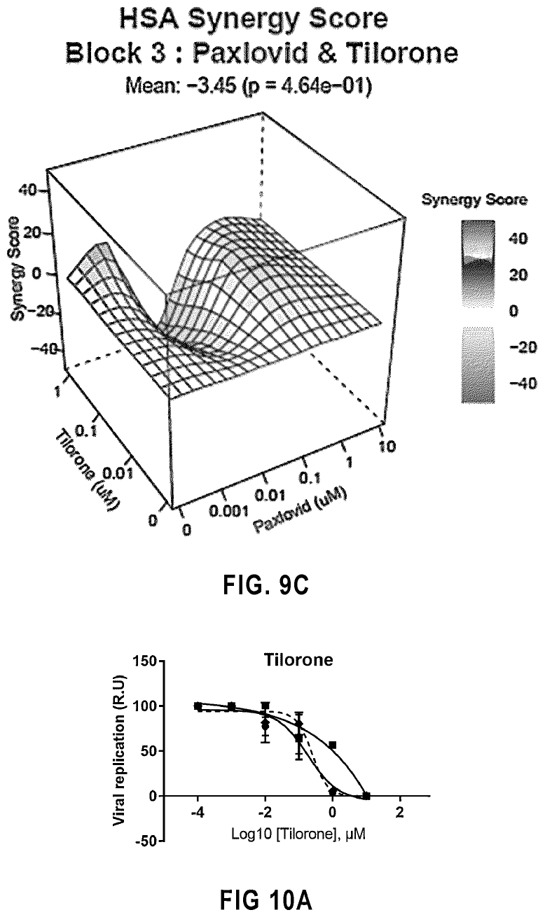

This step first involved implementing and validating coronavirus SARS-CoV-2 “checkerboard” assays to screen and test for synergy. This was implemented and data are demonstrated in Huh7.5 (FIG. 8) and Calu3 (FIG. 9).

In Huh7.5 cells, 4 combination with tilorone were tested (nelfinavir, molnupinavir, Paxlovid and trifluoperazine). Very promising results were obtained with nelfinavir-tilorone and molnupinavir-tilorone, for which synergy results were obtained with respective scores of 7.55 and 10.92. The other combinations, Paxlovid-tilorone and trifluoperazine-tilorone, give lower synergy scores with respective scores of 3.78 and-4.23. For Paxlovid-tilorone combination, synergy results were obtained when the cells were treated with high drug concentrations and an antagonistic profile was obtained when the cells were treated with low drug concentrations. For the trifluoperazine-tilorone combination, a high antagonism score (−4.23) was obtained. Therefore, combinations with trifluoperazine were not tested in Calu3 cells.

In Calu3 cells, 3 combinations with tilorone were tested (nelfinavir, molnupinavir and Paxlovid). Very promising results were obtained with nelfinavir-tilorone and molnupinavir-tilorone, for which positive synergy results were obtained with respective scores of 16.48 and 6.34. For the Paxlovid-tilorone combination, an antagonist result was obtained with the score of −3.48.

The results obtained in the Calu3 cells confirm those obtained in the Huh7.5 cells. As with the studies performed with rHCoVs-OC43, several more stringent synergy parameters were measured and collated in the tables below.

Table with synergy matrix results in Huh7.5.

| Drug 1 | Drug 2 | ZIP | Loewe | HSA | Bliss |
|---|---|---|---|---|---|
| Nelfinavir | Tilorone | −5.12 | 6.42 | 7.55 | −4.81 |
| Monulpinavir | | −0.84 | 3.88 | 10.92 | −1.93 |
| Paxlovid | | −8.62 | −4.38 | 3.78 | −10.33 |

Table with synergy matrix results in Calu3.

| Drug 1 | Drug 2 | ZIP | Loewe | HSA | Bliss |
|---|---|---|---|---|---|
| Nelfinavir | Tilorone | 9.66 | 16.78 | 16.48 | 7.77 |
| Monulpinavir | | 0.64 | 5.77 | 6.34 | 0.58 |
| Paxlovid | | −6.30 | −3.22 | −3.45 | −7.27 |

A toxicity measurement was carried out for the different combinations tested. Most combinations did not cause a greater toxic effect, except for the following combinations: tilorone and trifluoperazine (Huh7.5 cells) and tilorone-Paxlovid (Calu3). These combinations are not synergistic.

These results provide evidence that the following combinations synergistically inhibit SARS-CoV-2 replication:
nelfinavir+tilorone
tilorone+molnupivanir.

### Alpha and Beta Variants

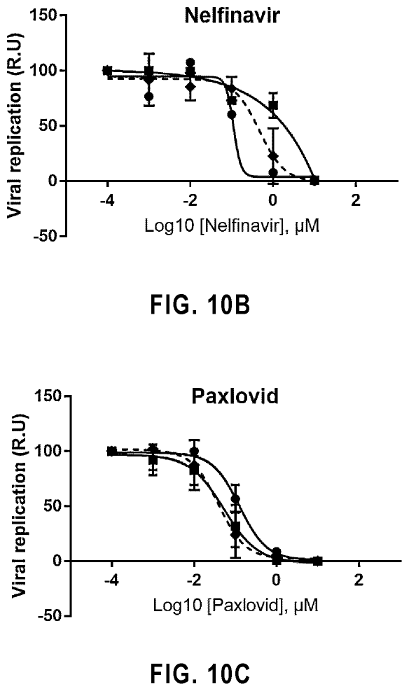

Tilorone, nelfinavir, molnupinavir and Paxlovid were tested as monotherapy on the alpha (British) and Beta (South African) variants. The results are summarized in the table below and shown in FIG. 10A-D.

| | SARS-CoV-2 | SARS-CoV-2 | SARS-CoV-2 |
|---|---|---|---|
| | (Vido) | (Alpha) | (Beta) |
| Drugs | EC₅₀ (μM) | EC₅₀ (μM) | EC₅₀ (μM) |
| Tilorone | 0.93 | 0.25 | 0.19 |
| Nelfinavir | 1.80 | 0.50 | 0.11 |
| Paxlovid | 0.05 | 0.04 | 0.13 |
| Molnupinavir | 0.09 | 0.06 | 0.11 |

### Example 3

### Step 4: Develop and Validate a Fast Synergy Screening Method to Promising Drug Combination Using Knew/Published Combination

Having determined the top candidate compounds as per Example 1, the next goal was to determine if combinations of the drugs would be synergistic, additive or antagonistic with regards to antiviral activity. As noted above, FIG. 3 shows diagrams of the two-part customized process that was implemented.

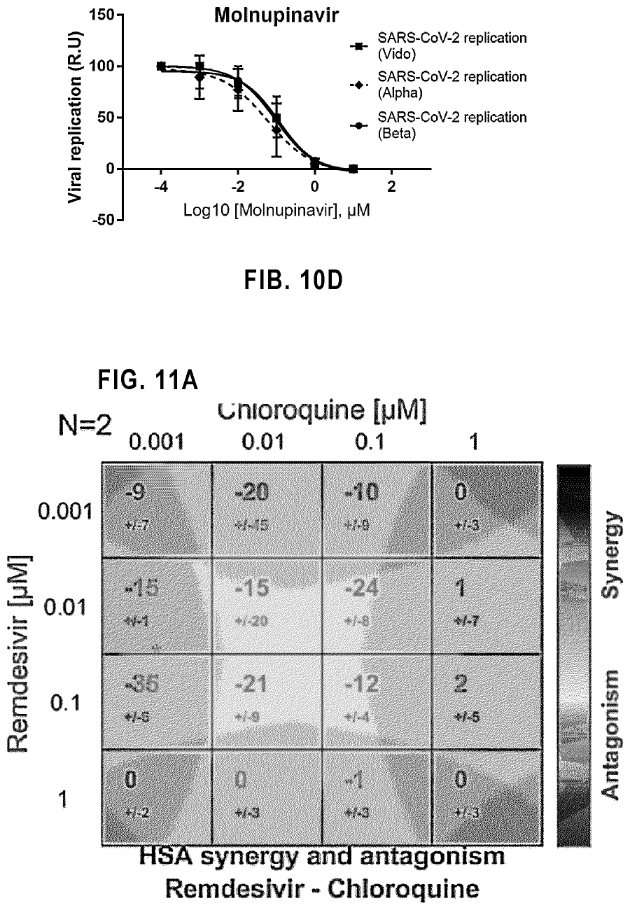

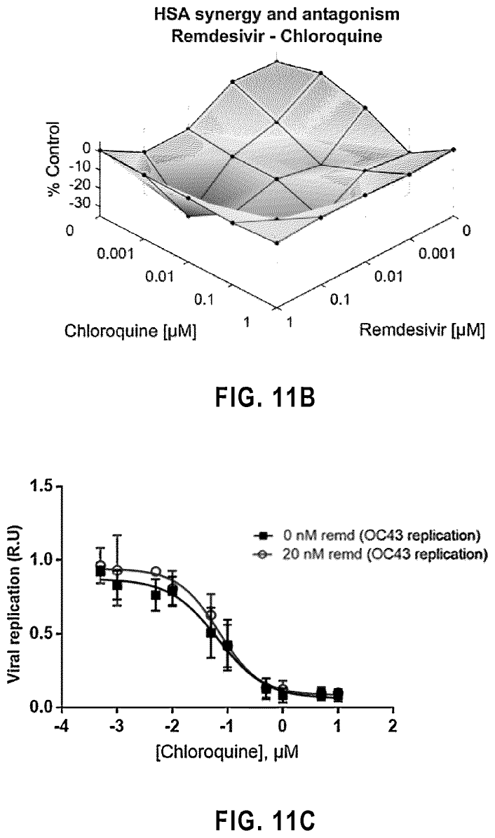

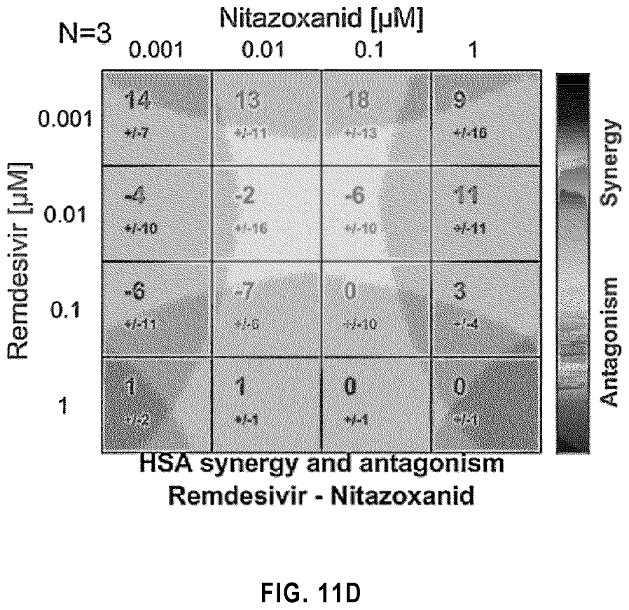

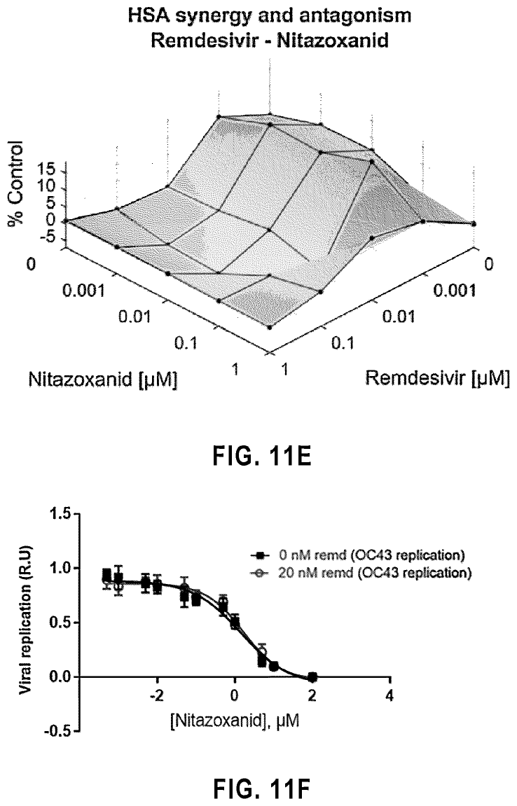

Data from Part 2 were used as input into the software Combenefit® which was coded by teams at Cambridge University and Astra Zeneca. A more detailed description of the Combenefit software is provided below. Overall, the software analyzes the input experimental values and compares them using mathematical equations (i.e., Bliss, Loewe and HSA models). The experimental dose-response surface data that delineates combination effects in concentration space, is first read by the software as a matrix of % of the control value across concentrations. Single agent effects are extracted from this data and fitted with a dose response curve. Based on the two single agent dose response curves, a model-based combination dose-response surface is derived. This surface provides a ‘reference’ dose-response surface for a non-synergistic (additive/independent) combination, whose characteristics are determined by the selected model (i.e., Bliss, Loewe, and HSA models). The experimental combination dose response surface is then compared to the model-generated one, resulting in a synergy distribution in concentration space. The dose-response data and analyses can be visualized as matrix format (FIGS. 11A and D), as surface display (FIGS. 11B and E), and/or as dose-response shifts (FIGS. 11C and F).

The software was put to a practical evaluation in partially controlled experiments. Based on literature reports, it was known that the combination of remdesivir and Chloroquine exhibited antagonistic effects on SAR-CoV-2 viral replication. Assuming that this may also be the case for rHCoVs-OC43, experiments were launched on rHCoVs-OC43 and data analyzed using the Combenefit software and visualizable in FIG. 11A-C. The valleys displayed in the surface view clearly show antagonistic effects as expected.

The software was put to another practical evaluation in partially controlled experiments. Based on literature reports, it was known that the combination of remdesivir and nitazoxanide exhibited synergic effects on SAR-CoV-2 viral replication. Assuming that this may also be the case for rHCoVs-OC43, experiments were launched on rHCoVs-OC43 and data analyzed using the Combenefit software and visualizable in FIG. 11D-F. The peaks in the surface view show synergic effects as could be expected from this partially controlled experiment.

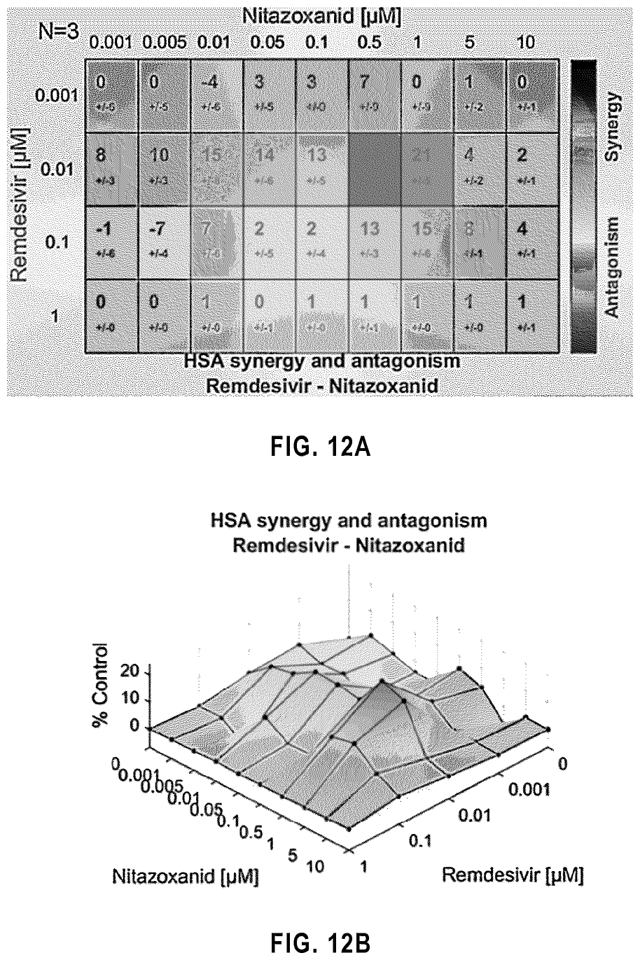

A modified and customized checkerboard method was tested using the same remdesivir and nitazoxanide combination as shown in FIG. 11D-E. This modified and customized checkerboard method involved an increase in the number of concentration points as shown at the bottom of FIGS. 12A and B. The peaks again clearly show synergistic effects. For the purposes of evaluation, the peaks represent synergistic data that brings inhibition above the control and is related to a high fractional inhibition concentration (FIC). Based on the above control studies, it was determined that the methods were now validated for probing combination effects.

### Step 5: Screening Drugs in Coronavirus rHCoVs-OC43 by Fast Synergy Method

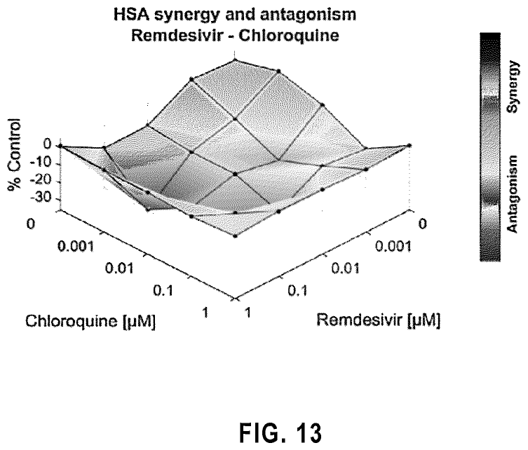

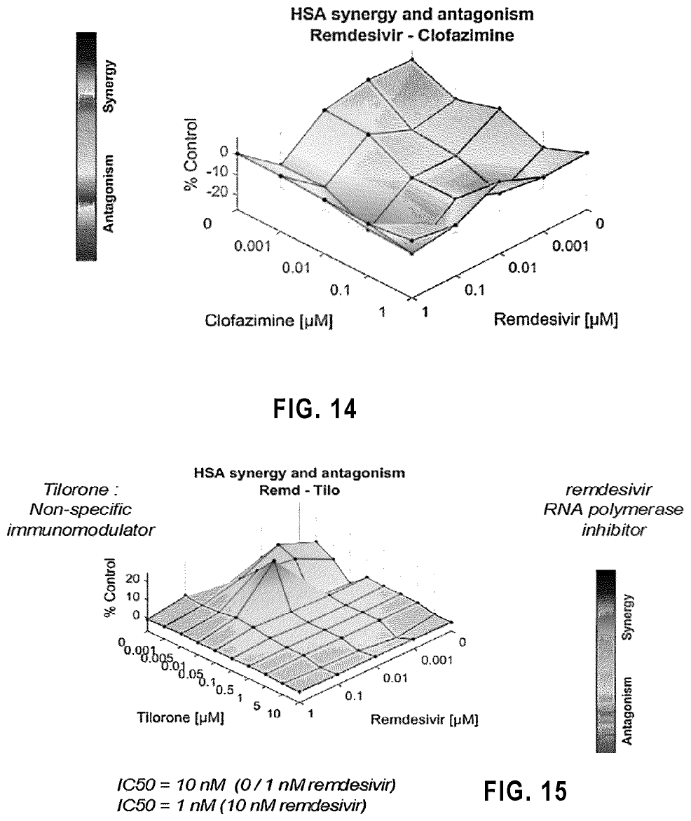

Step 5 then involved the screening of drugs in coronavirus rHCoVs-OC43 assays to select those that exhibited synergy effects. FIGS. 13 to 15 and the table below summarize results observed for remdesivir in combination with various drugs. Antagonistic and or additive effects were noted for one-to-one combinations of remdesivir with chloroquine, clofazimine, amodiaquine, vortioxetine nelfinavir, mycophenolate or toremifene. When an antagonism result was observed for one combination, it was designated a (−) and when a synergy result was observed it was designated a (+). When a mix of the two were noted, then it was designed “+/−” as a function of the HSA values obtained.

| Drug A | Drugs B | Synergy result |
|---|---|---|
| Remdesivir | Chloroquine, clofazimine | −− |
| | Amodiaquine, vortioxetine | − |
| | Nelfinavir, mycophenolate, toremifene, | −/+ |
| | Tilorone | + |
| | Nitazoxanide, | ++ |

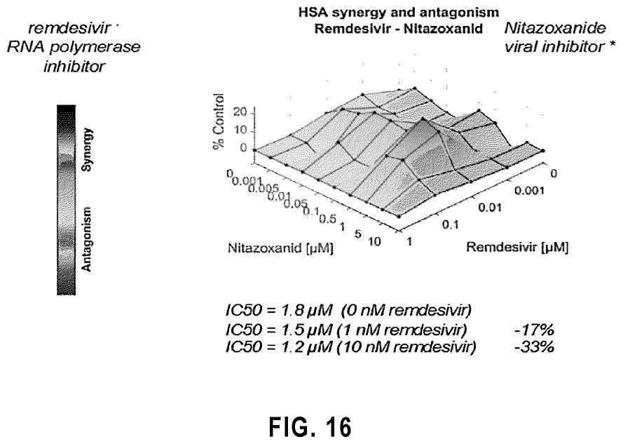

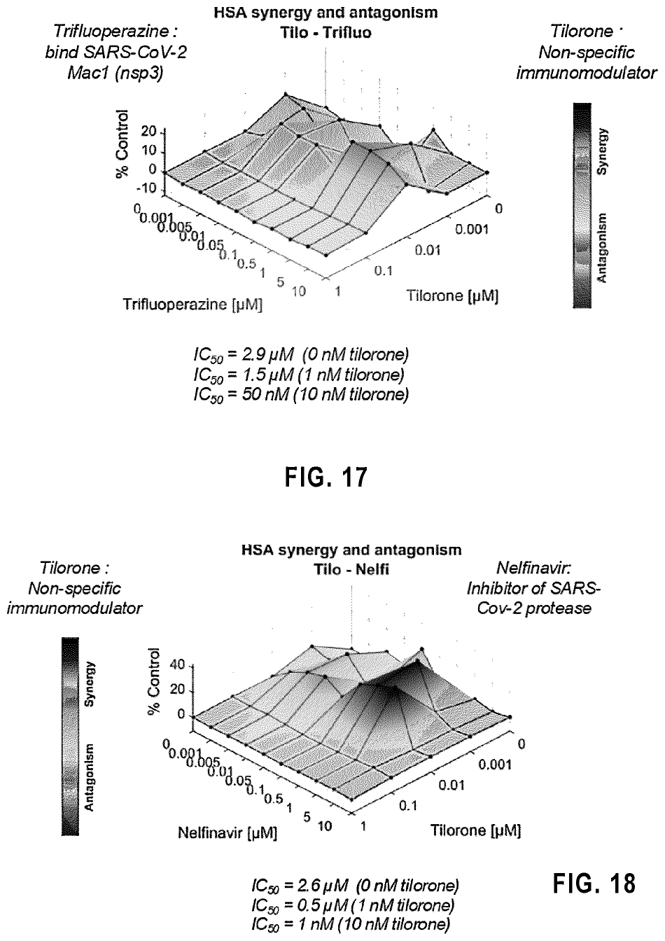

FIGS. 16 and 17 shows synergistic effects were noted for one-to-one combinations of remdesivir with tilorone or nitazoxanide. The EC₅₀ data show at the bottom report the quantitative data involving the synergistic effects. Thus, it is claimed here that combination drugs involving remdesivir with tilorone or remdesivir with nitazoxanide cold serve to reduce coronavirus replication and serve as medications for diseases related to coronaviruses.

Further studies were performed with alternate combinations of drugs. FIGS. 17 and 18 as well as the table below shows antagonistic effects were noted for one-to-one combinations of mycophenolate with nelfinavir or trifluoperazine. On the other hand, synergistic effects were noted for one-to-one combinations of tilorone with nelfinavir. The EC₅₀ data show at the bottom report the quantitative data involving the synergistic effects. Thus, it is claimed here that combination drugs involving tilorone with nelfinavir or trifluoperazine could serve to reduce coronavirus replication and serve as medications for diseases related to coronaviruses.

| Drug A | Drugs B | Synergy result |
|---|---|---|
| Tilorone | Trifluoperazine | ++ |
| | Nelfinavir | ++ |
| Mycophenolate | Nelfinavir | − |
| | Trifluoperazine | −− |

### Steps 6-7

Step 6 involved first implementing and validating coronavirus SARS-CoV-2 “checkerboard” assays to screen and test for synergy. This was implemented and data are demonstrated in FIG. 13.

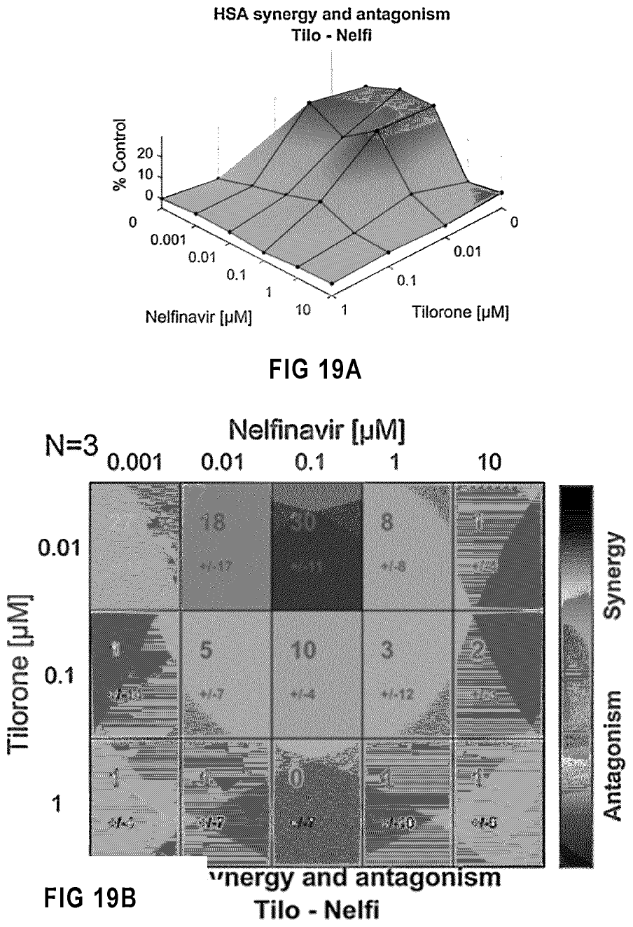

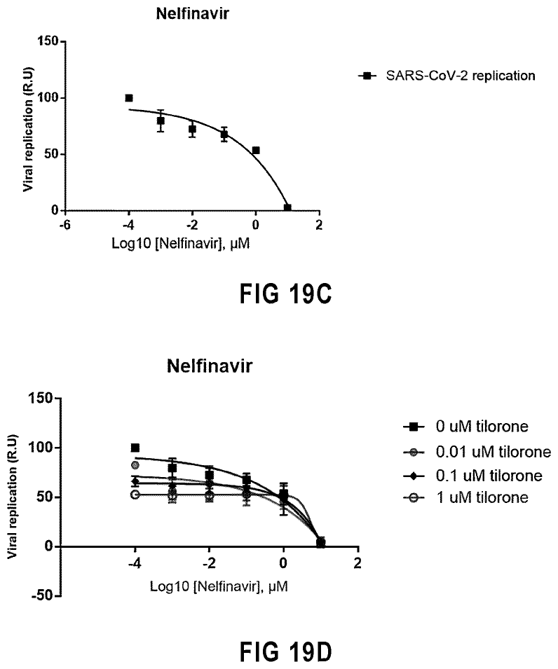

Step 7 involved screening drugs in coronavirus SARS-CoV-2 assays for to test for synergistic effects. FIG. 19A to D shows synergistic effects were noted for one-to-one combinations of tilorone with nelfinavir. The EC₅₀ data show the quantitative data involving the synergistic effects. Thus, it is claimed here that combination drugs involving tilorone with nelfinavir could serve to reduce coronavirus replication and serve as medications for diseases related to coronaviruses and specifically demonstrated here for SARS-CoV-2.

### Combenefit: Software Platform Used for the Analysis and Visualization of Drug Combinations

This software platform was used to analyze the drug combination data in Example 3. Here is a brief description of the theoretical aspects employed by the software.

### Synergy Distribution

Di Veroli et al. coded the software by considering the hypothetical combination of two agents A and B at concentrations a and b respectively. The effectiveness of a drug combination can be assessed in terms of the amount of “extra-effect” that is obtained when combining the drugs. Thus effects can be decomposed as following:

$$E ( a , b ) = R ( a , b ) + S ( a , b )$$

where E(a,b) is the observed effect, i.e. what is actually measured during the experiment. R(a,b) is the reference effect, often termed additive or independent (depending on the model used), i.e. a baseline which should be obtained in an experiment when the combination does not amplify or reduce cell kill. S(a,b) is the amount of extra-effect, also termed synergistic effect (when this extra-effect is negative, it is termed antagonistic). In order to identify synergy or antagonism, the combined reference effect R(a,b) is first derived based on single agent dose-response curves. Reference surfaces depend on the mathematical model which is used to define non-synergistic effects.

### Reference Models

The classical Loewe, Bliss and HSA models were implemented as follows.

### Loewe Model

For the Loewe model, the reference effect for the combination (a,b) is calculated by finding two doses a_{U} and b_{U} such that:

$${E}_{A} ( {a}_{u} ) = {E}_{B} ( {b}_{u} )$$

And for which the isobole equation is verified:

$$\frac{a}{{a}_{U}} + \frac{b}{{b}_{U}} = 1$$

These two equations are solved numerically for (aᵤ,bᵤ). The numerical solution is used to define the reference effect as:

$${R}_{AB \_ Loewe} ( a , b ) = E ( {a}_{u} ) = {E}_{B} ( {b}_{u} )$$

Because monotonic dose response curves are considered, if a solution exists, this solution is unique (i.e. the optimization process finds a global minimum). Note that, if for instance A has lower efficacy than B, then a solution does not exist for concentrations b high enough such that E_{B}(b)<min(E_{A}). Thus, applicability of the Loewe model is in principle limited.

For cases where the original isobole's equation of the Loewe model cannot be used due to differences in maximum effects, we developed an extension of the Loewe modelc as follows. Indeed, it can always be safely assumed that very high concentrations of A induce effects greater than observed efficacy (all substances are toxic at concentrations high enough). Thus, if we consider a concentration aᵤ which is great enough, then the ratio a/aᵤ becomes infinitely small and according to the isobole equation we obtain:

$$\frac{b}{{b}_{U}} = 1$$

Thus, for concentrations b that induces effects beyond A's observed maximum effect, we extend the Loewe model by defining the following reference:

$${R}_{AB \_ Loewe} ( a , b ) = E ( {b}_{u} ) = {E}_{B} ( b )$$

### Bliss Model

For the Bliss model, the reference effect for the combination (a,b) is obtained by taking the product of the effects at these concentrations:

$${R}_{AB \_ Bliss} ( a , b ) = {E}_{A} ( a ) \cdot {E}_{B} ( b )$$

In the software, the effect is defined as the fraction of unaffected control population (e.g. E(a=1 nM)=0.8, means that 1 nM of drug A reduces the expected population of cells by 20%—see also the online user's guide on how to prepare the data).

### HSA Model

For the HSA model, the reference effect for the combination (a,b) is obtained by taking the greatest effect (lowest residual compared to control) between the two drugs as single agents:

$${R}_{AB \_ HSA} ( a , b ) = MIN ( {E}_{A} ( a ) , {E}_{B} ( b ) )$$

### Synergy Metrics

Several metrics which summarize features of the synergy distribution are provided, as listed in the following table:

| | |
|---|---|
| Metric name | Metric formula |

Explanation of metric

| | |
|---|---|
| SYN_MAX | max(S(a, b)) |

This is the maximum level of synergy observed.

| | |
|---|---|
| ANT_MAX | min(S(a, b)) |

This is the maximum level of antagonism observed.

| | |
|---|---|
| SUM_SYN_ANT | ∫∫_{min(a>0),min(b>0)}^{max(a),max(b)}S(a, b) · dlog(a) · dlog(b) |

This is the sum of synergy and antagonism observed in concentration logarithmic space. For instance, an
integrated synergy of 50 is equivalent to an extra synergistic effect of 50% which is spread over a square
of 1 log × 1 log in the 2-d log-concentration space. Both antagonistic and synergistic effects are
considered in this metric.

| | |
|---|---|
| SUM_SYN_ANT_WEIGHTED | ∫∫_{min(a>0),min(b>0)}^{max(a),max(b)}(1 − E(a, b)/100) · S(a, b) · dlog(a) · dlog(b) |

The integrated weighted synergy incorporates a weight based on the dose response which bias the total
score towards synergy achieving highest effect. Hence, a synergy of 50% leading to a combined full
effect (100%) will have more weight than if the corresponding effect was only 20 or 30%. Both
antagonistic and synergistic effects are considered in this metric.

| | |
|---|---|
| SYN_SUM | ∫∫_{min(a>0), min(b>0)}^{max(a),max(b)}max(S(a, b), 0) · dlog(a) · dlog(b) |

Same as SUM_SYN_ANT, but antagonistic effects are not considered.

| | |
|---|---|
| SYN_SUM_WEIGHTED | ∫∫_{min(a>0),min(b>0)}^{max(a),max(b)}(1 − E(a, b)/100) · max(S(a, b), 0) · dlog(a) · dlog(b) |

Same as SUM_SYN_ANT_WEIGHTED, but antagonistic effects are not considered.

| | |
|---|---|
| ANT_SUM | ∫∫_{min(a>0),min(b>0)}^{max(a),max(b)}min(S(a, b), 0) · dlog(a) · dlog(b) |

Same as SUM_SYN_ANT, but synergistic effects are not considered.

| | |
|---|---|
| ANT_SUM_WEIGHTED | ∫∫_{min(a>0),min(b>0)}^{max(a),max(b)}(1 − E(a, b)/100) · min(S(a, b), 0) · dlog(a) · dlog(b) |

Same as SUM_SYN_ANT_WEIGHTED, but synergistic effects are not considered.

| | |
|---|---|
| SYN_SPREAD | \sqrt{SYN_SUM/SYN_MAX} |

A measure of synergy spread in logarithmic space. To differentiate for instance between a spread over a
square of 2 log × 2 log versus 1/2 log × 1/2 log.

| | |
|---|---|
| ANT_SPREAD | \sqrt{ANT_SUM/ANT_MAX} |

A similar measure of spread as SYN_SPREAD, but for antagonism.

| | |
|---|---|
| SYN_AVERAGE_C1 | 10∫∫_{min(a>0),min(b>0)}^{max(a),max(b)}loga · max(S(a,b),0) · dlog(a) · dlog(b)/SYN_SUM |

Concentration value of drug 1 where synergy appears to be localized.

| | |
|---|---|
| SYN_AVERAGE_C2 | 10∫∫_{min(a>0),min(b>0)}^{max(a),max(b)}logb · max(S(a,b),0) · dlog(a) · dlog(b)/SYN_SUM |

Concentration value of drug 2 where synergy appears to be localized.

| | |
|---|---|
| ANT_AVERAGE_C1 | 10∫∫_{min(a>0),min(b>0)}^{max(a),max(b)}loga · min(S(a,b),0) · dlog(a) · dlog(b)/SYN_SUM |

Concentration value of drug 1 where antagonism appears to be localized.

| | |
|---|---|
| ANT_AVERAGE_C2 | 10∫∫_{min(a>0),min(b>0)}^{max(a),max(b)}logb · max(S(a,b),0) · dlog(a) · dlog(b)/SYN_SUM |

Concentration value of drug 2 where antagonism appears to be localized.
log( ) refers to decimal logarithm

Once analyzed, the data can be visualized using a matrix synergy plot as described below. Contour plots are also available.

The scope of the claims should not be limited by the preferred embodiments set forth in the examples, but should be given the broadest interpretation consistent with the description as a whole.

### REFERENCES

The present description refers to a number of documents, the content of which is herein incorporated by reference in their entirety. These documents include, but are not limited to, the following:
V'kovski, P.; Kratzel, A.; Steiner, S.; Stalder, H.; Thiel, V., Coronavirus biology and replication: implications for SARS-CoV-2. Nature Reviews Microbiology 2021, 19 (3), 155-170.
Zhong, N. S.; Zheng, B. J.; Li, Y. M.; Poon; Xie, Z. H.; Chan, K. H.; Li, P. H.; Tan, S. Y.; Chang, Q.; Xie, J. P.; Liu, X. Q.; Xu, J.; Li, D. X.; Yuen, K. Y.; Peiris; Guan, Y., Epidemiology and cause of severe acute respiratory syndrome (SARS) in Guangdong, People's Republic of China, in February 2003. Lancet 2003, 362 (9393), 1353-8.
Ksiazek, T. G.; Erdman, D.; Goldsmith, C. S.; Zaki, S. R.; Peret, T.; Emery, S.; Tong, S.; Urbani, C.; Comer, J. A.; Lim, W.; Rollin, P. E.; Dowell, S. F.; Ling, A. E.; Humphrey, C. D.; Shieh, W. J.; Guarner, J.; Paddock, C. D.; Rota, P.; Fields, B.; DeRisi, J.; Yang, J. Y.; Cox, N.; Hughes, J. M.; LeDuc, J. W.; Bellini, W. J.; Anderson, L. J., A novel coronavirus associated with severe acute respiratory syndrome. N Engl J Med 2003, 348 (20), 1953-66.
van der Hoek, L.; Pyrc, K.; Jebbink, M. F.; Vermeulen-Oost, W.; Berkhout, R. J. M.; Wolthers, K. C.; Wertheim-van Dillen, P. M. E.; Kaandorp, J.; Spaargaren, J.; Berkhout, B., Identification of a new human coronavirus. Nature Medicine 2004, 10 (4), 368-373.
Zaki, A. M.; van Boheemen, S.; Bestebroer, T. M.; Osterhaus, A. D. M. E.; Fouchier, R. A. M., Isolation of a Novel Coronavirus from a Man with Pneumonia in Saudi Arabia. New England Journal of Medicine 2012, 367 (19), 1814-1820.
Zhu, N.; Zhang, D.; Wang, W.; Li, X.; Yang, B.; Song, J.; Zhao, X.; Huang, B.; Shi, W.; Lu, R., A novel coronavirus from patients with pneumonia in China, 2019. New England Journal of Medicine 2020.
Baden, L. R.; El Sahly, H. M.; Essink, B.; Kotloff, K.; Frey, S.; Novak, R.; Diemert, D.; Spector, S. A.; Rouphael, N.; Creech, C. B.; McGettigan, J.; Khetan, S.; Segall, N.; Solis, J.; Brosz, A.; Fierro, C.; Schwartz, H.; Neuzil, K.; Corey, L.; Gilbert, P.; Janes, H.; Follmann, D.; Marovich, M.; Mascola, J.; Polakowski, L.; Ledgerwood, J.; Graham, B. S.; Bennett, H.; Pajon, R.; Knightly, C.; Leav, B.; Deng, W.; Zhou, H.; Han, S.; Ivarsson, M.; Miller, J.; Zaks, T., Efficacy and Safety of the mRNA-1273 SARS-CoV-2 Vaccine. New England Journal of Medicine 2020, 384 (5), 403-416.
Polack, F. P.; Thomas, S. J.; Kitchin, N.; Absalon, J.; Gurtman, A.; Lockhart, S.; Perez, J. L.; Pérez Marc, G.; Moreira, E. D.; Zerbini, C.; Bailey, R.; Swanson, K. A.; Roychoudhury, S.; Koury, K.; Li, P.; Kalina, W. V.; Cooper, D.; Frenck, R. W.; Hammitt, L. L.; Türeci, Ö.; Nell, H.; Schaefer, A.; Ünal, S.; Tresnan, D. B.; Mather, S.; Dormitzer, P. R.; Şahin, U.; Jansen, K. U.; Gruber, W. C., Safety and Efficacy of the BNT 162b2 mRNA Covid-19 Vaccine. New England Journal of Medicine 2020, 383 (27), 2603-2615.
Pickard, A.; Calverley, B. C.; Chang, J.; Garva, R.; Gago, S.; Lu, Y.; Kadler, K. E., Discovery of re-purposed drugs that slow SARS-CoV-2 replication in human cells. PLOS Pathog. 2021, 17 (9), e1009840.
Riva, L.; Yuan, S.; Yin, X.; Martin-Sancho, L.; Matsunaga, N.; Pache, L.; Burgstaller-Muehlbacher, S.; De Jesus, P. D.; Teriete, P.; Hull, M. V.; Chang, M. W.; Chan, J. F.-W.; Cao, J.; Poon, V. K.-M.; Herbert, K. M.; Cheng, K.; Nguyen, T.-T. H.; Rubanov, A.; Pu, Y.; Nguyen, C.; Choi, A.; Rathnasinghe, R.; Schotsaert, M.; Miorin, L.; Dejosez, M.; Zwaka, T. P.; Sit, K.-Y.; Martinez-Sobrido, L.; Liu, W.-C.; White, K. M.; Chapman, M. E.; Lendy, E. K.; Glynne, R. J.; Albrecht, R.; Ruppin, E.; Mesecar, A. D.; Johnson, J. R.; Benner, C.; Sun, R.; Schultz, P. G.; Su, A. I.; Garcia-Sastre, A.; Chatterjee, A. K.; Yuen, K.-Y.; Chanda, S. K., Discovery of SARS-CoV-2 antiviral drugs through large-scale compound repurposing. Nature 2020, 586 (7827), 113-119.
Bakowski, M. A.; Beutler, N.; Wolff, K. C.; Kirkpatrick, M. G.; Chen, E.; Nguyen, T.-T. H.; Riva, L.; Shaabani, N.; Parren, M.; Ricketts, J.; Gupta, A. K.; Pan, K.; Kuo, P.; Fuller, M.; Garcia, E.; Teijaro, J. R.; Yang, L.; Sahoo, D.; Chi, V.; Huang, E.; Vargas, N.; Roberts, A. J.; Das, S.; Ghosh, P.; Woods, A. K.; Joseph, S. B.; Hull, M. V.; Schultz, P. G.; Burton, D. R.; Chatterjee, A. K.; McNamara, C. W.; Rogers, T. F., Drug repurposing screens identify chemical entities for the development of COVID-19 interventions. Nat. Commun. 2021, 12 (1), 3309.
Mslati, H.; Gentile, F.; Perez, C.; Cherkasov, A., Comprehensive Consensus Analysis of SARS-CoV-2 Drug Repurposing Campaigns. J. Chem. Inf. Mod. 2021, 61 (8), 3771-3788.
Xiao, X.; Wang, C.; Chang, D.; Wang, Y.; Dong, X.; Jiao, T.; Zhao, Z.; Ren, L.; Dela Cruz, C. S.; Sharma, L.; Lei, X.; Wang, J., Identification of Potent and Safe Antiviral Therapeutic Candidates Against SARS-CoV-2. Frontiers in Immunology 2020, 11 (2877).
Siegel, D.; Hui, H. C.; Doerffler, E.; Clarke, M. O.; Chun, K.; Zhang, L.; Neville, S.; Carra, E.; Lew, W.; Ross, B.; Wang, Q.; Wolfe, L.; Jordan, R.; Soloveva, V.; Knox, J.; Perry, J.; Perron, M.; Stray, K. M.; Barauskas, O.; Feng, J. Y.; Xu, Y.; Lee, G.; Rheingold, A. L.; Ray, A. S.; Bannister, R.; Strickley, R.; Swaminathan, S.; Lee, W. A.; Bavari, S.; Cihlar, T.; Lo, M. K.; Warren, T. K.; Mackman, R. L., Discovery and Synthesis of a Phosphoramidate Prodrug of a Pyrrolo[2,1-f][triazin-4-amino] Adenine C-Nucleoside (GS-5734) for the Treatment of Ebola and Emerging Viruses. J. Med. Chem. 2017, 60 (5), 1648-1661.
Singh, A. K.; Singh, A.; Singh, R.; Misra, A., Remdesivir in COVID-19: A critical review of pharmacology, pre-clinical and clinical studies. Diabetes & Metabolic Syndrome: Clinical Research & Reviews 2020, 14 (4), 641-648.
Choy, K.-T.; Wong, A. Y.-L.; Kaewpreedee, P.; Sia, S. F.; Chen, D.; Hui, K. P. Y.; Chu, D. K. W.; Chan, M. C. W.; Cheung, P. P.-H.; Huang, X.; Peiris, M.; Yen, H.-L., Remdesivir, lopinavir, emetine, and homoharringtonine inhibit SARS-CoV-2 replication in vitro. Antiviral Research 2020, 178, 104786.
Cao, B.; Wang, Y.; Wen, D.; Liu, W.; Wang, J.; Fan, G.; Ruan, L.; Song, B.; Cai, Y.; Wei, M.; Li, X.; Xia, J.; Chen, N.; Xiang, J.; Yu, T.; Bai, T.; Xie, X.; Zhang, L.; Li, C.; Yuan, Y.; Chen, H.; Li, H.; Huang, H.; Tu, S.; Gong, F.; Liu, Y.; Wei, Y.; Dong, C.; Zhou, F.; Gu, X.; Xu, J.; Liu, Z.; Zhang, Y.; Li, H.; Shang, L.; Wang, K.; Li, K.; Zhou, X.; Dong, X.; Qu, Z.; Lu, S.; Hu, X.; Ruan, S.; Luo, S.; Wu, J.; Peng, L.; Cheng, F.; Pan, L.; Zou, J.; Jia, C.; Wang, J.; Liu, X.; Wang, S.; Wu, X.; Ge, Q.; He, J.; Zhan, H.; Qiu, F.; Guo, L.; Huang, C.; Jaki, T.; Hayden, F. G.; Horby, P. W.; Zhang, D.; Wang, C., A Trial of Lopinavir-Ritonavir in Adults Hospitalized with Severe Covid-19. New England Journal of Medicine 2020, 382 (19), 1787-1799.
Vandyck K, Deval J (August 2021). “Considerations for the discovery and development of 3-chymotrypsin-like cysteine protease inhibitors targeting SARS-CoV-2 infection”. Current Opinion in Virology. 49:36-40. doi:10.1016/j.coviro.2021.04.006. PMC 8075814. PMID 34029993.
Şimşek-Yavuz S, Komsuoǧlu Çelikyurt FI (August 2021). “Antiviral treatment of COVID-19: An update”. Turkish Journal of Medical Sciences. doi:10.3906/sag-2106-250. PMID 34391321. S2CID 237054672.
Ahmad B, Batool M, Ain QU, Kim MS, Choi S (August 2021). “Exploring the Binding Mechanism of PF-07321332 SARS-CoV-2 Protease Inhibitor through Molecular Dynamics and Binding Free Energy Simulations”. International Journal of Molecular Sciences. 22 (17): 9124. doi:10.3390/ijms22179124. PMC 8430524. PMID 34502033.
“Pfizer Announces Additional Phase 2/3 Study Results Confirming Robust Efficacy of Novel COVID-19 Oral Antiviral Treatment Candidate in Reducing Risk of Hospitalization or Death” (Press release). 14 Dec. 2021.
Toots M, Yoon JJ, Cox RM, Hart M, Sticher ZM, Makhsous N, et al. (October 2019). “Characterization of orally efficacious influenza drug with high resistance barrier in ferrets and human airway epithelia”. Science Translational Medicine. 11 (515): eaax5866. doi:10.1126/scitranslmed.aax5866. PMC 6848974. PMID 31645453.
Toots M, Yoon JJ, Hart M, Natchus MG, Painter GR, Plemper RK (April 2020). “Quantitative efficacy paradigms of the influenza clinical drug candidate EIDD-2801 in the ferret model”. Translational Research. 218: 16-28. doi:10.1016/j.trsl.2019.12.002. PMC 7568909. PMID 31945316.
Combenefit—https://www.cruk.cam.ac.uk/research-groups/jodrell-group/combenefit Di Veroli GY, Fornari C, Wang D, Mollard S, Bramhall JL, Richards FM, Jodrell DI. Combenefit: an interactive platform for the analysis and visualization of drug combinations. Bioinformatics. 2016 Sep. 15; 32(18):2866-8.
Wang, M.; Cao, R.; Zhang, L.; Yang, X.; Liu, J.; Xu, M.; Shi, Z.; Hu, Z.; Zhong, W.; Xiao, G., Remdesivir and chloroquine effectively inhibit the recently emerged novel coronavirus (2019-nCOV) in vitro. Cell Research 2020, 30 (3), 269-271.
Shyr, Z.A.; Gorshkov, K.; Chen, C.Z. & Zheng, W., Drug Discovery Strategies for SARS-CoV-2, Journal of Pharmacology and Experimental Therapeutics October 2020, 375 (1) 127-138.

1. A combination of drugs comprising:
A) tilorone or a pharmaceutically acceptable salt thereof and B) nelfinavir or a pharmaceutically acceptable salt thereof,
A) tilorone or a pharmaceutically acceptable salt thereof and B) molnupinavir or a pharmaceutically acceptable salt thereof,
A) tilorone or a pharmaceutically acceptable salt thereof and B) remdesivir or a pharmaceutically acceptable salt thereof,
A) tilorone or a pharmaceutically acceptable salt thereof and B) alfacalcidol or a pharmaceutically acceptable salt thereof,
A) tilorone or a pharmaceutically acceptable salt thereof and B) clofazimine or a pharmaceutically acceptable salt thereof,
A) tilorone or a pharmaceutically acceptable salt thereof and B) danazol or a pharmaceutically acceptable salt thereof,
A) tilorone or a pharmaceutically acceptable salt thereof and B) manidipine or a pharmaceutically acceptable salt thereof,
A) tilorone or a pharmaceutically acceptable salt thereof and B) sertraline or a pharmaceutically acceptable salt thereof,
A) tilorone or a pharmaceutically acceptable salt thereof and B) trifluoperazine or a pharmaceutically acceptable salt thereof,
A) remdesivir or a pharmaceutically acceptable salt thereof and B) hexachlorophene or a pharmaceutically acceptable salt thereof,
A) remdesivir or a pharmaceutically acceptable salt thereof and B) mycophenolate or a pharmaceutically acceptable salt thereof,
A) mycophenolate or a pharmaceutically acceptable salt thereof and B) hexachlorophene or a pharmaceutically acceptable salt thereof,
A) mycophenolate or a pharmaceutically acceptable salt thereof and B) nelfinavir or a pharmaceutically acceptable salt thereof, or
A) mycophenolate or a pharmaceutically acceptable salt thereof and B) nitazoxanide or a pharmaceutically acceptable salt thereof.

2. The combination of claim 1, comprising:
A) tilorone or a pharmaceutically acceptable salt thereof and B) nelfinavir or a pharmaceutically acceptable salt thereof,
A) tilorone or a pharmaceutically acceptable salt thereof and B) molnupinavir or a pharmaceutically acceptable salt thereof, or
A) tilorone or a pharmaceutically acceptable salt thereof and B) remdesivir or a pharmaceutically acceptable salt thereof.

3. The combination of claim 2, comprising:
A) tilorone or a pharmaceutically acceptable salt thereof and B) nelfinavir or a pharmaceutically acceptable salt thereof, or
A) tilorone or a pharmaceutically acceptable salt thereof and B) molnupinavir or a pharmaceutically acceptable salt thereof.

4. The combination of claim 1, comprising (A) tilorone or a pharmaceutically acceptable thereof and (B) nelfinavir or a pharmaceutically acceptable thereof.

5. The combination of claim 1, comprising (A) tilorone or a pharmaceutically acceptable thereof and (B) remdesivir or a pharmaceutically acceptable thereof.

6. The combination of claim 1, comprising (A) tilorone or a pharmaceutically acceptable thereof and (B) molnupinavir or a pharmaceutically acceptable thereof.

7. The combination of claim 1, comprising A) tilorone or a pharmaceutically acceptable salt thereof and B) alfacalcidol or a pharmaceutically acceptable salt thereof,

8. The combination of claim 1, comprising A) tilorone or a pharmaceutically acceptable salt thereof and B) clofazimine or a pharmaceutically acceptable salt thereof,

9. The combination of claim 1, comprising A) tilorone or a pharmaceutically acceptable salt thereof and B) danazol or a pharmaceutically acceptable salt thereof,

10. The combination of claim 1, comprising A) tilorone or a pharmaceutically acceptable salt thereof and B) manidipine or a pharmaceutically acceptable salt thereof,

11. The combination of claim 1, comprising A) tilorone or a pharmaceutically acceptable salt thereof and B) sertraline or a pharmaceutically acceptable salt thereof,

12. The combination of claim 1, comprising A) tilorone or a pharmaceutically acceptable salt thereof and B) trifluoperazine or a pharmaceutically acceptable salt thereof,

13. The combination of claim 1, comprising A) remdesivir or a pharmaceutically acceptable salt thereof and B) hexachlorophene or a pharmaceutically acceptable salt thereof,

14. The combination of claim 1, comprising A) remdesivir or a pharmaceutically acceptable salt thereof and B) mycophenolate or a pharmaceutically acceptable salt thereof,

15. The combination of claim 1, comprising A) mycophenolate or a pharmaceutically acceptable salt thereof and B) hexachlorophene or a pharmaceutically acceptable salt thereof,

16. The combination of claim 1, comprising A) mycophenolate or a pharmaceutically acceptable salt thereof and B) nelfinavir or a pharmaceutically acceptable salt thereof, or

17. The combination of claim 1, comprising A) mycophenolate or a pharmaceutically acceptable salt thereof and B) nitazoxanide or a pharmaceutically acceptable salt thereof.

18. The combination of any one or claims 1 to 17, wherein the pharmaceutically acceptable salt of nelfinavir is nelfinavir methanesulfonate.

19. The combination of any one of claims 1 to 17, wherein the pharmaceutically acceptable salt of tilorone is tilorone dihydrochloride.

20. The combination of any one of claims 1 to 17, wherein the pharmaceutically acceptable salt of manidipine is manidipine dihydrochloride.

21. The combination of any one of claims 1 to 17, wherein the pharmaceutically acceptable salt of sertraline is sertraline hydrochloride.

22. The combination of any one of claims 1 to 17, wherein the pharmaceutically acceptable salt of the trifluoperazine is trifluoperazine dihydrochloride.

23. The combination of any one of claims 1 to 17, wherein the pharmaceutically acceptable salt of mycophenolate is mycophenolate mofetil.

24. The combination of any one of claims 1 to 23, wherein components (a) and (b) are formulated in a same pharmaceutical composition.

25. The combination of any one of claims 1 to 23, wherein components (a) and (b) are formulated in different pharmaceutical compositions.

26. A pharmaceutical composition comprising the combination of any one of claims 1 to 20 and one or more pharmaceutically acceptable excipient.

27. A method for blocking the replication of a coronavirus in a coronavirus-infected cell, comprising contacting the cell with an effective amount of the combination of any one of claims 1 to 26.

28. A method for treating an infection by a coronavirus in a subject comprising administering to said subject an effective amount of the combination of any one of claims 1 to 26.

29. A method for treating a coronavirus-related disease in a subject comprising administering to said subject an effective amount of the combination of any one of claims 1 to 26.

30. A method for reducing the risk of developing a coronavirus-related disease or the severity of a coronavirus-related disease in a subject comprising administering to said subject an effective amount of the combination of any one of claims 1 to 26.

31. The method of any one of claims 27 to 30, wherein the coronavirus is a beta-coronavirus.

32. The method of claim 31, wherein the beta-coronavirus is SARS-CoV-2.

33. The method of claim 29, wherein the coronavirus-related disease is COVID-19.

34. The method of any one of claims 28 to 30, wherein the subject is a human.

35. Use of the combination of any one of claims 1 to 26, for the manufacture of a medicament for blocking the replication of a coronavirus in a coronavirus-infected cell.

36. Use of the combination of any one of claims 1 to 26, for the manufacture of a medicament for treating an infection by a coronavirus in a subject.

37. Use of the combination of any one of claims 1 to 26, for the manufacture of a medicament for treating a coronavirus-related disease in a subject.

38. Use of the combination of any one of claims 1 to 26, for the manufacture of a medicament for reducing the risk of developing a coronavirus-related disease or the severity of a coronavirus-related disease in a subject.

39. The use of any one of claims 35 to 38, wherein the coronavirus is a beta-coronavirus.

40. The use of claim 39, wherein the beta-coronavirus is SARS-CoV-2.

41. The use of claim 38, wherein the coronavirus-related disease is COVID-19.

42. The use of any one of claims 36 to 38, wherein the subject is a human.

43. The combination of any one of claims 1 to 26 for use in blocking the replication of a coronavirus in a coronavirus-infected cell.

44. The combination of any one of claims 1 to 26 for use in treating an infection by a coronavirus in a subject.

45. The combination of any one of claims 1 to 26 for use in treating a coronavirus-related disease in a subject.

46. The combination of any one of claims 1 to 26 for use in reducing the risk of developing a coronavirus-related disease or the severity of a coronavirus-related disease in a subject.

47. The combination for use according to any one of claims 43 to 46, wherein the coronavirus is a beta-coronavirus.

48. The combination for use according to claim 47, wherein the beta-coronavirus is SARS-CoV-2.

49. The combination for use according to claim 45, wherein the coronavirus-related disease is COVID-19.

50. The combination for use according to any one of claims 44 to 46, wherein the subject is a human.
